(19) United States
(12) Patent Application Publication
(10) Pub. No.: US 2011/0111025 A1
(43) Pub. Date: May 12, 2011

(54) High Drug Load Formulations and Dosage Forms

(75) Inventors: Gaylen M. Zentner, Salt Lake City, UT (US); James C. McRea, Salt Lake City, UT (US); Mark S. Williams, Salt Lake City, UT (US); Gregory T. Oerhtman, Overland Park, KS (US); Tracy Annette Powers, Saint Louis, MO (US)

(73) Assignee: Myriad Pharmaceuticals, Inc., Salt Lake City, UT (US)

(21) Appl. No.: 12/771,621

(22) Filed: Apr. 30, 2010

Related U.S. Application Data
(63) Continuation of application No. 11/491,771, filed on Jul. 24, 2006, now abandoned.
(60) Provisional application No. 60/701,710, filed on Jul. 22, 2005.
(60) Provisional application No. 60/706,344, filed on Aug. 8, 2005.

(51) Int. Cl. A61K 31/192; A61K 9/28; A61P 25/28

(52) U.S. Cl. 424/465; 514/570

(57) ABSTRACT

The invention relates to high drug load formulations containing (R)-2-(2-fluoro-4-biphenylyl)propionic acid as an active pharmaceutical ingredient.

### CROSS REFERENCE TO RELATED APPLICATIONS

This application is a continuation of U.S. application Ser. No. 11/491,771, filed Jul. 24, 2006, which itself claims priority under 35 U.S.C. §119(e) to U.S. provisional application Nos. 60/701,710, filed Jul. 22, 2005 and 60/706,344 filed Aug. 8, 2005, all of which are incorporated herein by reference in their entireties.

### FIELD OF THE INVENTION

The invention relates to pharmaceutical formulations and dosage forms, particularly pharmaceutical formulations having a high drug load.

### BACKGROUND OF THE INVENTION

A particularly difficult problem facing the pharmaceutical and medical communities is patient compliance with dosing regimens. Lack of adherence to a dosing regimen can be disastrous. Generally speaking, depending on the pharmacokinetic and pharmacodynamic behavior of a specific therapeutic, and the nature of the disease, a drug concentration profile in a target tissue must be achieved to produce a therapeutic effect. Efficacious drug concentration profiles are achieved through patient compliance with dosing regimens that were shown to produce a clinically relevant effect during controlled clinical trials.

Non-compliance (non-adherence) with a prescribed dosing regimen has negative clinical consequences. Lack of compliance can result in lower levels of drug in the target tissue and the disease may “escape” the effects of the drug since it is not present at inhibitory concentrations. For example, non-compliance with the prescribed treatment regimen for antiretroviral medication has led to drug-resistant HIV strains which have been transmitted throughout the population (Boden et al. JAMA 282:1135-1141 (1999)). In fact, there is a steep drop in sustaining viral load as compliance goes from 95% to 70% (Paterson et al. Ann. Int. Med. 133:21-30 (2000)), resulting in more problems for the patient.

The pharmaceutical/medical community has focused drug development clinical trials on simple dosing regimens to promote compliance. Drugs that require complex dosing regimens are now routinely abandoned because patients will not or can not comply with the required dosing regimen. One interesting example of how compliance and tablet size affect drug development is the story of the protease inhibitors amprenavir and fosamprenavir.

Amprenavir was approved by the FDA in 1999 as an HIV protease inhibitor but never gained widespread use because it was found to be significantly less effective than indinavir. In fact, current guidelines recommend against using amprenavir due to high tablet burden and its potential effect on compliance (Panel on Clinical Practices for the Treatment of HIV infection, convened by the Department of Health and Human Services. Guidelines for the use of antiretroviral agents in HIV-infected adults and adolescents. See www.Aidsinfo.nih.gov). Recently fosamprenavir was approved by the FDA for the treatment of HIV-1. Fosamprenavir is a prodrug of amprenavir that has improved solubility and oral bioavailability, allowing for once or twice daily dosing with smaller tablets and fewer tablets than amprenavir, leading to increased patient acceptance of the fosamprenavir dosing regimen and the expected greater compliance and clinical efficacy.

One particular problem for creating acceptable dosing regimens is when large amounts of a drug need to be delivered to a patient: there is a limit to the size of a tablet a patient is able to swallow and the more tablets a patient has to take the more likely they will make a mistake, resulting in non-compliance. There is a need for formulations useful for the delivery of large amounts of drug, with manageable tablet (pill) burden and acceptable tablet size.

Formulation of pharmaceutical tablets typically involves mixing the active pharmaceutical ingredient (API; the drug) with one or more inactive ingredients (i.e., excipients). Tablets that contain low doses (e.g., less than 50 mg drug per dose) will often be formulated with more excipient on a weight basis than the API to facilitate the manufacturing process (e.g., compaction), yet still result in small tablets that are easy for the patient to swallow. Since the excipient comprises a substantial portion of the total tablet weight, the processing and manufacturability of the tablets are readily adjusted regardless of the properties of the drug agent.

Conversely, with high dose drugs, the characteristics of the tablet are strongly influenced by the properties of the API. If these properties are not compatible with commercial manufacturing requirements, the formulator is faced with producing tablets that are larger in size (adding excipients to solve the manufacturing problems), or requiring the administration of multiple tablets, each containing a lowered percentage of API, both of which negatively impact patient compliance.

There is a need for (R)-2-(2-fluoro-4-biphenylyl)propionic acid containing dosage forms that contain a high drug load where the drug comprises a high percentage of the total dosage form weight to provide dosage forms of a size that facilitates patient compliance with common dosing regimens.

### BRIEF SUMMARY OF THE INVENTION

The invention relates to high drug load formulations having (R)-2-(2-fluoro-4-biphenylyl)propionic acid as the active pharmaceutical ingredient. The inventors have discovered formulations of (R)-2-(2-fluoro-4-biphenylyl)propionic acid that allow for the production of (R)-2-(2-fluoro-4-biphenylyl)propionic acid containing tablets each having 200 mg or more of API, excellent mechanical properties and dissolution profiles, and therapeutically desirable pharmacokinetic profiles. The inventive high drug load formulations allow for the production of tablets having 55% or more (by weight) (R)-2-(2-fluoro-4-biphenylyl)propionic acid. The inventive compositions also have desirable manufacturing characteristics. The high drug load formulations are suited for use in conditions requiring the dosing of high levels of (R)-2-(2-fluoro-4-biphenylyl)propionic acid, like Alzheimer's disease.

In a first embodiment, the invention provides a pharmaceutical composition having (R)-2-(2-fluoro-4-biphenylyl)propionic acid or a pharmaceutically acceptable salt thereof admixed with one or more pharmaceutically acceptable excipients, where the weight of (R)-2-(2-fluoro-4-biphenylyl)propionic acid is 55% or more of the total weight of the unit dosage form. In some aspects of this embodiment, (R)-2-(2-fluoro-4-biphenylyl)propionic acid can be 57% or more, 60% or more, or 63% or more of the total weight of the unit dosage form. In some aspects of this embodiment, the unit dosage form has about 200 mg, 200 or more mg, 300 mg, 300 or more mg, 400 mg, 400 or more mg, 500 mg, 500 or more mg, 600 mg, 600 or more mg, 700 mg, 700 or more mg, 800 mg, or 800 or more mg (R)-2-(2-fluoro-4-biphenylyl)propionic acid. In one aspect of this embodiment, the (R)-2-(2-fluoro-4-biphenylyl)propionic acid formulation has a disintegrant as an ingredient (e.g., microcrystalline cellulose and/or croscarmellose sodium). In another aspect of this embodiment, the (R)-2-(2-fluoro-4-biphenylyl)propionic acid formulation has a binder as an ingredient (e.g., hydroxypropyl methylcellulose). In another aspect of this embodiment, the (R)-2-(2-fluoro-4-biphenylyl)propionic acid formulation has a diluent as an ingredient (e.g., lactose). In another aspect of this embodiment, the (R)-2-(2-fluoro-4-biphenylyl)propionic acid formulation has a glidant as an ingredient (e.g., colloidal silicon dioxide). In another aspect of this embodiment, the (R)-2-(2-fluoro-4-biphenylyl)propionic acid formulation has a lubricant as an ingredient (e.g., magnesium stearate). The formulations and unit dosage forms of this embodiment of the invention, optionally, can have coatings, coloring agents, stabilizers, preservatives, and/or flavoring agents.

The formulation of this embodiment can be provided as a unit dosage form suited for oral administration (e.g., a tablet). The first embodiment of the invention further provides a (R)-2-(2-fluoro-4-biphenylyl)propionic acid formulation having from 55% to 90% by weight (R)-2-(2-fluoro-4-biphenylyl)propionic acid and from 10% to 45% by weight inactive pharmaceutical ingredients. In one aspect, the formulation has from 55% to 85% by weight (R)-2-(2-fluoro-4-biphenylyl)propionic acid and 15%-45% by weight inactive pharmaceutical ingredients. In one aspect, the formulation has from 55% to 75% by weight (R)-2-(2-fluoro-4-biphenylyl)propionic acid and from 25% to 45% by weight inactive ingredients. In one aspect, the formulation has from 60% to 70% by weight (R)-2-(2-fluoro-4-biphenylyl)propionic acid and from 30% to 40% by weight inactive pharmaceutical ingredients.

In a second embodiment, the invention provides a tablet dosage form having between 320 to 480 mg (R)-2-(2-fluoro-4-biphenylyl)propionic acid, or a pharmaceutically acceptable salt thereof, where the long axis of the tablet is from about 0.6 to 0.8 inches, and the tablet width is from about 0.3 to 0.4 inches. In one aspect of this embodiment, the unit dosage form is no longer than 0.82 inches, no longer than 0.80 inches, no longer than 0.77 inches, no longer than 0.72 inches, or no longer than 0.70 inches. In one aspect of this embodiment, the unit dosage form is no wider than 0.41 inches, no wider than 0.40 inches, no wider than 0.38 inches, or no wider than 0.35 inches. In some aspects of the invention, the total volume of the unit dosage form is less than 0.70 cm³, less than 0.65 cm³, less than 0.60 cm³, less than 0.55 cm³, less than 0.50 cm³, or less than 0.45 cm³. In some aspects of this embodiment, each tablet has one or more excipients chosen from disintegrants, binders, diluents, glidants, lubricants, coloring agents, stabilizers, preservatives, and/or flavoring agents. In some aspects of this embodiment, each tablet has (R)-2-(2-fluoro-4-biphenylyl)propionic acid and one or more binders, one or more diluents, one or more disintegrants, one or more glidants, one or more lubricants, and if desired, one or more optional ingredients. In one aspect of this embodiment, the tablet unit dosage form is coated.

In a third embodiment, the invention provides a tablet unit dosage form having 55% or more by weight of (R)-2-(2-fluoro-4-biphenylyl)propionic acid, or a pharmaceutically acceptable salt thereof, and that yields a dissolution profile substantially similar to one or more of those shown in FIGS. 1 and 2. The unit dosage form of this embodiment is suited for oral administration. In a related embodiment, the unit dosage form is a capsule dosage form. In one aspect of this third embodiment, the unit dosage form has (R)-2-(2-fluoro-4-biphenylyl)propionic acid and one or more pharmaceutically acceptable excipients. With a tablet dosage form, the one or more excipients can be chosen from disintegrants, binders, diluents, glidants, lubricants, coloring agents, stabilizers, preservatives, and/or flavoring agents. In one aspect of this embodiment, the unit dosage form is a coated tablet. In other aspects of this embodiment, the unit dosage form has (R)-2-(2-fluoro-4-biphenylyl)propionic acid and one or more pharmaceutically acceptable excipients in amounts sufficient to yield a dissolution profile substantially similar to one or more of those shown in FIGS. 1 and 2. In one aspect of this embodiment, a dosage form is provided having between 320 to 480 mg (R)-2-(2-fluoro-4-biphenylyl)propionic acid that yields a dissolution profile substantially similar to one or more of those shown in FIGS. 1 and 2.

In a fourth embodiment, the invention provides a tablet unit dosage form containing about 400 mg (R)-2-(2-fluoro-4-biphenylyl)propionic acid, or a pharmaceutically acceptable salt thereof, and having 55% or more by weight of (R)-2-(2-fluoro-4-biphenylyl)propionic acid in the tablet. According to this embodiment, the 400 mg unit dosage form yields a pharmacokinetic profile that is substantially similar (bioequivalent) to that shown in FIG. 3 after oral administration of the indicated dose to a fasting individual (e.g., two tablets, each containing 400 mg (R)-2-(2-fluoro-4-biphenylyl)propionic acid for 800 mg total of API). The unit dosage form of this embodiment is suited for oral administration. In a related embodiment, the unit dosage form is a capsule dosage form.

In one aspect of this fourth embodiment, the unit dosage form has (R)-2-(2-fluoro-4-biphenylyl)propionic acid and one or more pharmaceutically acceptable excipients as components. With a tablet dosage form the one or more excipients can be chosen from disintegrants, binders, diluents, glidants, lubricants, coloring agents, stabilizers, preservatives, and/or flavoring agents. In one aspect of this fourth embodiment, the unit dosage form is a coated tablet. In another aspect of this embodiment, the unit dosage form has (R)-2-(2-fluoro-4-biphenylyl)propionic acid and one or more pharmaceutically acceptable excipients in amounts sufficient to yield a pharmacokinetic profile substantial similar (bioequivalent) to that shown in FIG. 3 when administered orally to a fasting individual. In one aspect of this embodiment, a dosage form is provided having between 320 to 480 mg (R)-2-(2-fluoro-4-biphenylyl)propionic acid that yields a pharmacokinetic profile substantially similar (bioequivalent) to that shown in FIG. 3 for the 800 mg BID dose group (e.g., 2 tablets each having about 400 mg (R)-2-(2-fluoro-4-biphenylyl)propionic acid).

In one aspect, the unit dosage form according to any of the embodiments of the invention, when tested in pH 7.2 potassium phosphate buffer at 37° C. using a USP Apparatus 2 (paddles), at a rotation speed of 75 rpm, has a dissolution profile such that less than 85, 80, 75, 70, 65, or 60 weight percent of the (R)-2-(2-fluoro-4-biphenylyl)propionic acid is released at 15 minutes. In one aspect, the unit dosage form according to any of the embodiments of the invention, when tested in pH 7.2 potassium phosphate buffer at 37° C. using a USP Apparatus 2 (paddles), at a rotation speed of 75 rpm, has a dissolution profile such not that less than 3, 5, 10, 15, 20, 30, 40, 50, 60, 70, or 80 weight percent of the (R)-2-(2-fluoro-4-biphenylyl)propionic acid is released at 15 minutes. In another aspect, the unit dosage form according to any of the embodiments of the invention, when tested in pH 7.2 potassium phosphate buffer at 37° C. using a USP Apparatus 2 (paddles), at a rotation speed of 75 rpm, has a dissolution profile such that greater than 80, 85, 90, or 95 weight percent of the (R)-2-(2-fluoro-4-biphenylyl)propionic acid is released at 45 minutes. In another aspect, the unit dosage form according to any of the embodiments of the invention, when tested in pH 7.2 potassium phosphate buffer at 37° C. using a USP Apparatus 2 (paddles), at a rotation speed of 75 rpm, has a dissolution profile such that greater than 70, 80, 90, 92, 94, or 96 weight percent of the (R)-2-(2-fluoro-4-biphenylyl)propionic acid is released at 60 minutes.

In another embodiment, the invention provides compositions and methods useful for preparing unit dosage forms having (R)-2-(2-fluoro-4-biphenylyl)propionic acid, or a pharmaceutically acceptable salt thereof, as the active pharmaceutical ingredient. According to one aspect of this embodiment of the invention, the composition is a pre-blend composition having (R)-2-(2-fluoro-4-biphenylyl)propionic acid, one or more diluents, and one or more glidants as ingredients. In some aspects of this embodiment, (R)-2-(2-fluoro-4-biphenylyl)propionic acid is present in the pre-blend composition in amounts from 50-95%, 60-95%, or 70-95% of the total weight of the pre-blend composition. The diluent is present in an amount sufficient to allow for adequate mixing with the other formulation ingredients and/or allow for adequate flowability during manufacturing. In some aspects of this embodiment, the pre-blend composition has one or more diluents present in amounts from 1-30%, 3-25%, or 5-20% of the total weight of the pre-blend composition. The glidant is present in an amount sufficient to insure adequate flow qualities of the powdered mixture (pre-blend composition). In some aspects of this embodiment, the pre-blend composition has one or more glidants present in amounts from 0.01-5%, 0.1-5%, or 0.1-3% of the total weight of the pre-blend composition. In some aspects of this embodiment, optional ingredients are present in amounts from 0-20%, 1-20%, or 1-10% of the total weight of the pre-blend composition. In one aspect, the method includes charging (R)-2-(2-fluoro-4-biphenylyl)propionic acid, the one or more diluents, the one or more glidants and any optional ingredients in a blender followed by blending for an amount of time sufficient to provide a substantially uniform pre-blend composition. The pre-blend composition can then be used in the next step of the process—milling. According to one aspect of the invention, the pre-blend composition is then milled through a screen having a size sufficient to reduce the particle size of the larger particles of the pre-blend composition to give a milled composition. The milled composition can then be used to form a wet granulation. In an alternative aspect, the materials can be charged directly into the high shear granulator and blended in the high shear granulator prior to the wet granulation step.

In yet another embodiment, the invention provides a wet granulation composition having (R)-2-(2-fluoro-4-biphenylyl)propionic acid, one or more diluents, one or more binders, one or more glidants, one or more wetting agents and optionally, one or more additional ingredients. Furthermore, this embodiment provides a method for wet granulation of the wet granulation composition. In one aspect of this embodiment, (R)-2-(2-fluoro-4-biphenylyl)propionic acid is present in the wet granulation composition in amounts from 40-95%, 45-95%, or 50-90% of the total weight of the wet granulation composition. In one aspect of this embodiment, the one or more diluents are present in the wet granulation composition in amounts from 1-30%, 1-20%, or 5-15% of the total weight of the wet granulation composition. In one aspect of this embodiment, the one or more glidants are present in the wet granulation composition in amounts from 0.01-10%, 0.01-5%, or 0.1-5% of the total weight of the wet granulation composition. The binder is present in an amount sufficient to impart an immediate release dissolution profile for the coated tablet unit dosage form. In one aspect of this embodiment, the one or more binders are present in the wet granulation composition in amounts from 1-30%, 1-20%, or 1-15% of the total weight of the wet granulation composition. The wetting agent is present in an amount sufficient to avoid the formation of granules that are hard enough to require excessive pressure to tablet (and/or prevent the formation of tablets having a mottled appearance) and/or result in granules that are overly soft causing difficulties during compression (and/or break down during lubrication). In one aspect of this embodiment, the one or more wetting agents are present in the wet granulation composition in amounts from 1-40%, 1-25%, or 5-25% of the total weight of the wet granulation composition. In some aspects of this embodiment, optional ingredients are present in amounts from 0-20%, 1-20%, or 1-10% of the total weight of the wet granulation composition. According to one aspect of this embodiment, the binder and milled composition are charged into a granulator and dry blended for an amount of time sufficient to adequately mix/blend the milled composition and the binder (e.g., provide a substantially uniform mixture), followed by high shear granulation with the wetting agent for an amount of time sufficient to result in the formation of distinct granules. The wet granulation is then milled through a screen size sufficient reduce the median size of the particles. The wet granulation is then dried by a method appropriate form removing the wetting agent. The “dried” wet granulation can then be milled through a screen size sufficient to yield a granulation composition (component) having a suitable particle size.

In still another embodiment, the invention provides a pre-tablet composition having a granulation component and one or more disintegrants. The pre-tablet composition has (R)-2-(2-fluoro-4-biphenylyl)propionic acid, one or more diluents, one or more binders, one or more glidants, one or more disintegrants, and optionally, one or more optional ingredients. In one aspect of this embodiment, (R)-2-(2-fluoro-4-biphenylyl)propionic acid is present in the pre-tablet composition in amounts from 50-95%, 55-90%, or 55-85% of the total weight of the pre-tablet composition. In one aspect of this embodiment, the one or more diluents are present in the pre-tablet composition in amounts from 1-30%, 1-20%, or 5-15% of the total weight of the pre-tablet composition. In one aspect of this embodiment, the one or more binders are present in the pre-tablet composition in amounts from 1-30%, 1-20%, or 1-15% of the total weight of the pre-tablet composition. In one aspect of this embodiment, the one or more glidants are present in the pre-tablet composition in amounts from 0.01-10%, 0.01-5%, or 0.1-5% of the total weight of the pre-tablet composition. The disintegrant is present in an amount sufficient to yield an immediate release dissolution profile of the unit dosage form. In one aspect of this embodiment, the one or more disintegrants are present in the pre-tablet composition in amounts from 1-40%, 5-25%, or 5-20% of the total weight of the pre-tablet composition. In one aspect of this embodiment, one or more optional ingredients are present in the pre-tablet composition in amounts from 1-20%, 1-25%, or 5-25% of the total weight of the pre-tablet composition. According to one aspect of this embodiment, the dried granulation is charged into a blender along with the one or more disintegrants and any optional/additional ingredients followed by blending for an amount of time to provide a substantially uniform mixture.

In one embodiment, the invention provides a tableting composition having the pre-tableting composition and one or more lubricants. Thus, this embodiment relates to formulations having (R)-2-(2-fluoro-4-biphenylyl)propionic acid as an API, one or more diluents, one or more binders, one or more glidants, one or more disintegrants, and one or more lubricants, and methods of preparing such compositions. The composition of this embodiment is suited for compression tableting. In one aspect of this embodiment, (R)-2-(2-fluoro-4-biphenylyl)propionic acid is present in the pre-tableting composition in amounts from 50-95%, 55-90%, or 55-85% of the total weight of the tableting composition. In one aspect of this embodiment, the one or more diluents are present in the tableting composition in amounts from 1-30%, 1-20%, or 5-15% of the total weight of the tableting composition. In one aspect of this embodiment, the one or more binders are present in the tableting composition in amounts from 1-30%, 1-20%, or 1-15% of the total weight of the tableting composition. In one aspect of this embodiment, the one or more glidants are present in the tableting composition in amounts from 0.01-10%, 0.01-5%, or 0.1-5% of the total weight of the tableting composition. In one aspect of this embodiment, the one or more disintegrants are present in the tableting composition in amounts from 1-40%, 5-25%, or 5-20% of the total weight of the tableting composition. The lubricant is present in an amount sufficient to allow ejection of the tablet cleanly from the die with minimal stress to the tablet. In one aspect of this embodiment, the one or more lubricants are present in amounts from 0.01-10%, 0.1-10%, or 0.1-5% of the total weight of the composition of this embodiment. In some aspects of this embodiment, the composition is prepared by charging the one or more lubricants into the diffusion blender with the other components (e.g., those in embodiment three) and blending for an amount of time sufficient to yield a substantially uniform mixture. The composition prepared according to this embodiment can then be compressed into tablets with an appropriate press. The composition is sufficiently compressed to yield a tablet that, when coated, yields an immediate release dissolution profile that is substantial similar to one or more of those shown in FIG. 1 and FIG. 2.

In another embodiment, the invention relates to preparing a coated tablet having (R)-2-(2-fluoro-4-biphenylyl)propionic acid as an API. The tablet (i.e., those prepared according to the previous embodiment) is coated with a coating sufficient to yield an immediate release dissolution profile of the coated tablet unit dosage form and/or to impart sufficient stability to the unit dosage form (e.g., meets United States Pharmacopeial (USP) standards). According one aspect of this embodiment, a film coating suspension is prepared with a suitable coating agent and water. The film coating suspension can then used to coat the tablets in, e.g., a perforated coating pan to yield a coated tablet. In some aspects of this embodiment, the coating represents from 0.1-15%, 0.1-10%, or 1-7% of the total weight of the tablet.

In yet another embodiment, the invention provides an (R)-2-(2-fluoro-4-biphenylyl)propionic acid tablet unit dosage form produced according to the methods of the invention that yields a dissolution profile substantial similar to one or more of those shown in FIGS. 1 and 2. In another aspect of this embodiment, the invention provides an (R)-2-(2-fluoro-4-biphenylyl)propionic acid tablet unit dosage form produced according to the methods of the invention that yields a pharmacokinetic profile substantially similar to that shown in FIG. 3 for the indicated dose.

In some aspects of the invention, the method of making the tablet unit dosage forms of the invention involves a high shear wet granulation process. In a specific aspect of the invention, the general scheme for the process of making the unit dosage form of the invention involves producing a pre-blend composition having (R)-2-(2-fluoro-4-biphenylyl)propionic acid, one or more diluents, and one or more glidants, that is blended for an amount of time sufficient to give a uniform pre-mill composition. The pre-mill composition is then used in the next step of the process—milling. The milled composition is next used to form a wet granulation. One or more binders and the milled composition are then charged into a granulator and dry blended, followed by granulation with the wetting agent to give the wet granulation. The wet granulation is then milled, dried, and then milled again to give the intra-granular portion of the unit dosage form. Next, one or more disintegrants are added to the intra-granular component and blended in a diffusion blender. Next, the composition is prepared by charging one or more lubricants into the diffusion blender with the other components. This composition is then ready for tableting with a compression tableter. After formation of the tablets, they then can be coated to give the unit dosage form. One exemplary method of this aspect of the invention is outlined in FIG. 4.

In some aspects of the above-described embodiments, the unit dosage form of the invention can be manufactured using a high shear granulation process in which (R)-2-(2-fluoro-4-biphenylyl)propionic acid is pre-blended and pre-milled with one or more binders such as lactose (e.g., anhydrous) and one or more glidants such as colloidal silicon dioxide. The pre-blend can be processed in a drum blender followed by milling to decrease the median particle size of the large particles of the (R)-2-(2-fluoro-4-biphenylyl)propionic acid pre-blend prior to high shear granulation. Once granulated, the pre-blend can be dried, milled, blended, compressed on a high-speed rotary press and coated in a perforated pan.

The formulations and unit dosage forms of the invention are useful for treating diseases and conditions where high levels of (R)-2-(2-fluoro-4-biphenylyl)propionic acid need to be delivered to the patient. In some aspects, the invention provides a method of using the unit dosage form as in any of the embodiments of the invention comprising identifying an individual in need of treatment and administering to said individual a therapeutically effective amount the unit dosage form. In some aspects of the invention, the individual in need of treatment has a neurodegenerative disorder. In some aspects of the invention, the neurodegenerative disorder is chosen from Alzheimer's disease, dementia, mild cognitive impairment, Parkinson's disease, Huntington's disease and symptoms thereof. In some aspects of the invention the individual in need of treatment has a form of Alzheimer's disease chosen from prodromal Alzheimer's disease, mild Alzheimer's disease, mild-to-moderate Alzheimer's disease, moderate Alzheimer's disease, moderate-to-severe Alzheimer's disease, severe Alzheimer's disease, dementia and/or vascular dementia. In some aspects of the invention, the individual in need of treatment has a form of Alzheimer's disease which is mild Alzheimer's disease. In some aspects of this embodiment, the individual in need of treatment is at risk for developing Alzheimer's disease or desires prophylaxis against the onset of Alzheimer's disease. In some aspect of the invention, the unit dosage form is administered twice daily (e.g., two 400 mg tablets in the morning and two 400 mg tablets in the evening). In some aspects of the invention, the unit dosage form comprises from about 320 to 480 mg of (R)-2-(2-fluoro-4-biphenylyl)propionic acid or molar equivalent of a pharmaceutically acceptable salt thereof and the individual is administered two unit dosage forms twice daily (e.g., two unit dosage forms in the morning and two unit dosage forms in the evening). In some aspects of the invention, the individual in need of treatment has Alzheimer's disease or desires prophylaxis against the development of symptoms of Alzheimer's disease. In some aspects of the invention, the individual in need of treatment has cancer. In some aspects of the invention, the individual in need of treatment has a cancer (or is seeking prevention of a cancer) chosen from brain, lung, liver, spleen, kidney, lymph node, small intestine, pancreas, blood cell, colon, stomach, breast, endometrial, prostate, testicle, ovary, skin, and head and neck cancer, esophagus, and bone marrow cancer. In one aspect, the individual in need of treatment has prostate cancer. Skilled artisans are capable of identifying individuals in need of treatment.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, examples of suitable methods and materials are described below. In case of conflict, the present specification, including definitions, will control. In addition, the materials, methods, and examples are illustrative only and are not intended to be limiting.

Other features and advantages of the invention will be apparent from the following detailed description, and from the claims.

### BRIEF DESCRIPTION OF THE DRAWINGS

FIG. 1 illustrates the dissolution profiles of various (R)-2-(2-fluoro-4-biphenylyl)propionic acid containing tablets having a PVP binder. See Example 3 for experimental details.

FIG. 2 illustrates the dissolution profiles of various (R)-2-(2-fluoro-4-biphenylyl)propionic acid containing tablets having a HPMC binder. See Example 3 for experimental details.

FIG. 3 represents a PK profile favorable for the treatment of disorders where the concentration of (R)-2-(2-fluoro-4-biphenylyl)propionic acid much must be maintained at a therapeutic level over sustained periods. These profiles were obtained under conditions where the individuals had been on a BID dosing regimen (e.g., 800_BID refers to 800 mg of API twice daily) for a period of time sufficient to achieve steady state drug concentrations prior to taking the indicated dose (200 mg API, 400 mg API, or 800 mg API) after fasting.

FIG. 4 is an exemplary flow chart illustrating a process of the invention.

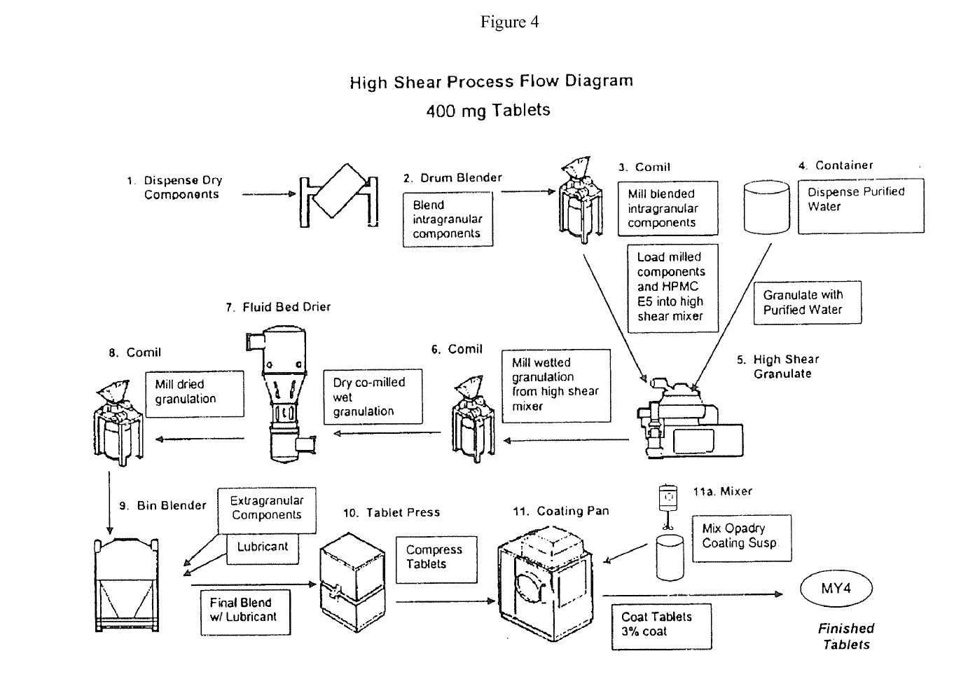

### DETAILED DESCRIPTION OF THE INVENTION

The invention relates to a high drug load pharmaceutical formulation having (R)-2-(2-fluoro-4-biphenylyl)propionic acid as the active pharmaceutical ingredient. The invention encompasses oral compositions that provide pharmaceutical, pharmacokinetic, and therapeutic characteristics particularly useful in treating and preventing Alzheimer's disease, prostate cancer, as well as other disorders. The composition of the invention is formulated with one or more pharmaceutically acceptable excipients (inactive pharmaceutical ingredients). The pharmaceutical composition of the invention is formulated for oral administration (e.g., a tablet dosage form). The (R)-2-(2-fluoro-4-biphenylyl)propionic acid containing composition of the invention can be used in methods for treating, preventing (delaying the onset of one or more symptoms of a disease), and prophylaxis against neurodegenerative disorders such as Alzheimer's disease, or neoplastic diseases such as prostate cancer. The inventors have discovered formulations of (R)-2-(2-fluoro-4-biphenylyl)propionic acid that allow for the production of (R)-2-(2-fluoro-4-biphenylyl)propionic acid tablets having 200 mg or more of API, excellent manufacturing properties, mechanical properties, dissolution profiles, and therapeutically desirable pharmacokinetic profiles. The inventive formulations allow for the production of tablets having 55% or more by weight of the active pharmaceutical ingredient.

The invention also relates to composition and processes for making pharmaceutical compositions that exhibit one or more superior properties relative to other compositions comprising (R)-2-(2-fluoro-4-biphenylyl)propionic acid. These superior properties include, but are not limited to, one or more of the following: improved bioavailability, improved solubility of the pharmaceutical composition, improved disintegration times for immediate release oral dosage forms, improved dissolution times for immediate release oral dosage forms, decreased tablet friability, increased tablet hardness, improved safety for oral dosage forms, reduced moisture content and/or hygroscopicity for oral dosage forms, improved composition wettability, improved particle size distribution of (R)-2-(2-fluoro-4-biphenylyl)propionic acid, improved composition compressibility, improved composition flow properties, improved chemical stability of the final oral dosage form, improved physical stability of the final oral dosage form, decreased tablet size, improved blend uniformity, improved dose uniformity, increased granule density for wet granulated compositions, reduced water requirements for wet granulation, reduced wet granulation time, and/or reduced drying time for wet granulated mixtures.

The formulations and unit dosage forms of the invention contain (R)-2-(2-fluoro-4-biphenylyl)propionic acid as an active pharmaceutical ingredient. (R)-2-(2-fluoro-4-biphenylyl)propionic acid is the “R” enantiomer of flurbiprofen ((R,S)-2-(2-fluoro-4-biphenylyl)propionic acid). (R)-2-(2-fluoro-4-biphenylyl)propionic acid can be obtained from resolving racemic flurbiprofen or through enantioselective or enantiospecific syntheses. The R-enantiomer of flurbiprofen ((R)-2-(2-fluoro-4-biphenylyl)propionic acid), or a desired enantiomeric excess of (R)-2-(2-fluoro-4-biphenylyl)propionic acid, can be obtained by resolving the racemic flurbiprofen according to well-known methods, and is also commercially available (e.g., Caymen Chemical, Ann Arbor, Mich.). Methods of resolving (R)-2-(2-fluoro-4-biphenylyl)propionic acid from the racemate are disclosed in U.S. Pat. No. 5,599,969 to Hardy et al. which discloses reacting racemic flurbiprofen with α-methylbenzylamine to form an isolatable salt of (R)-2-(2-fluoro-4-biphenylyl)propionic acid. U.S. Pat. No. 4,209,638 to Boots Co. discloses a process for resolving 2-arylpropionic acids, which include flurbiprofen, by mixing the racemate with a chiral organic nitrogenous base under certain conditions followed by recovery and separation of the diastereomeric salts. Other patents disclosing processes for resolving racemic arylpropionic acids include U.S. Pat. No. 4,983,765 to PAZ; U.S. Pat. No. 5,015,764 to Ethyl Corp.; U.S. Pat. No. 5,235,100 to Ethyl Corp.; U.S. Pat. No. 5,574,183 to Albemarle Corp.; and U.S. Pat. No. 5,510,519 to Sumitomo Chemical Company.

The oral unit dosage forms of the present invention can contain any of the following inactive ingredients, or compounds of a similar nature: a diluent such as lactose; a binder such as hydroxypropyl methylcellulose; a disintegrating agent (disintegrant) such as croscarmellose sodium, or microcrystalline cellulose; a lubricant such as magnesium stearate or stearic acid; a glidant such as colloidal silicon dioxide; and optional ingredients such as coloring agents, stabilizers, preservatives and/or flavoring agents. In addition, dosage forms of the invention can contain various other materials which modify the physical form of the dosage unit, for example, polymeric coatings (e.g., cellulosics, methacrylates, or acrylates), sugar coatings, shellac coatings, color coatings, wax coatings, or other types of coatings.

The invention provides pharmaceutical compositions having (R)-2-(2-fluoro-4-biphenylyl)propionic acid and one or more pharmaceutically acceptable excipients, with (R)-2-(2-fluoro-4-biphenylyl)propionic acid comprising 55% or more of the total weight of the unit dosage form. The unit dosage form of this embodiment is suited for oral administration (e.g., a tablet). In some aspects of this embodiment, (R)-2-(2-fluoro-4-biphenylyl)propionic acid is 57% or more, 60% or more, or 63% or more of the total weight of the of the unit dosage form. In some aspects of this embodiment, the unit dosage form has about 200 mg, 200 or more mg, 300 mg, 300 or more mg, 400 mg, 400 or more mg, 800 mg, and 800 or more mg (R)-2-(2-fluoro-4-biphenylyl)propionic acid in the free acid form. (R)-2-(2-fluoro-4-biphenylyl)propionic acid can be present in the formulation as the free acid form, or as a salt form of the free acid (percentages and weights given in reference to the free acid throughout unless otherwise noted; salt form weights and percentages are calculated based on having the same molar equivalent as the free acid). In one specific aspect of this embodiment, approximately 400 mg of (R)-2-(2-fluoro-4-biphenylyl)propionic acid free acid is present in a tablet formulation and comprises from 65% to 68% of the total weight of the tablet.

The invention provides an (R)-2-(2-fluoro-4-biphenylyl)propionic acid pharmaceutical formulation having from 55% to 90% by weight (R)-2-(2-fluoro-4-biphenylyl)propionic acid and from 10% to 45% by weight inactive pharmaceutical ingredients. The formulation can be a unit dosage form suited for oral administration (e.g., a tablet). In one aspect of the invention, the (R)-2-(2-fluoro-4-biphenylyl)propionic acid formulation has from 55% to 85% by weight (R)-2-(2-fluoro-4-biphenylyl)propionic acid and from 15% to 45% by weight inactive pharmaceutical ingredients. In another aspect of the invention, the (R)-2-(2-fluoro-4-biphenylyl)propionic acid formulation has from 55% to 75% by weight (R)-2-(2-fluoro-4-biphenylyl)propionic acid and from 25% to 45% inactive ingredients. In another aspect, the (R)-2-(2-fluoro-4-biphenylyl)propionic acid formulation has from 60% to 70% by weight (R)-2-(2-fluoro-4-biphenylyl)propionic acid and from 30% to 40% inactive pharmaceutical ingredients. According to one specific aspect of this embodiment, the formulation has from 55% to 90% by weight (R)-2-(2-fluoro-4-biphenylyl)propionic acid, 1% to 20% by weight lactose (calculated based on anhydrous lactose), 1% to 20% by weight hydroxypropyl methylcellulose, 5% to 45% by weight microcrystalline cellulose, and, if desired, optional ingredients.

The (R)-2-(2-fluoro-4-biphenylyl)propionic acid containing pharmaceutical formulations of the present invention generally have 55% or more of the total weight of the unit dosage form as (R)-2-(2-fluoro-4-biphenylyl)propionic acid, with the remaining weight comprised of one or more pharmaceutically acceptable excipients. The excipients for use in the formulations and unit dosage forms of the invention include one or more excipients chosen from disintegrants, binders, diluents, glidants, and lubricants, as well as any desired optional ingredient. Thus, in one aspect of the invention, the unit dosage form has an excipient that is a disintegrant (e.g., microcrystalline cellulose and/or croscarmellose). The amount of disintegrant in the pharmaceutical formulation can be 45% or less, 40% or less, 35% or less, 30% or less, or less than 25% of the total weight of the unit dosage form. In another aspect of the invention, the unit dosage form has an excipient that is a binder (e.g., hydroxypropyl methylcellulose). The amount of binder in the pharmaceutical formulation can be 20% or less, 15% or less, 10% or less, or less than 8% of the total weight of the unit dosage form. In yet another aspect of the invention, the unit dosage form has an excipient that is a diluent such as lactose. The amount of diluent in the pharmaceutical formulation can be 20% or less, 17% or less, 15% or less, or less than 12% of the total weight of the unit dosage form. In still another aspect of the invention, the unit dosage form has an excipient that is a glidant such as colloidal silicon dioxide. The amount of glidant in the pharmaceutical formulation can be 7% or less, 5% or less, 3% or less, or less than 2% of the total weight of the unit dosage form. In another aspect of the invention, the unit dosage form has an excipient that is a lubricant such as magnesium stearate. The amount of lubricant in the pharmaceutical formulation can be 10% or less, 5% or less, 3% or less, or less than 2% of the total weight of the unit dosage form. In another aspect of the invention, the unit dosage form, containing (R)-2-(2-fluoro-4-biphenylyl)propionic acid and one or more excipients, is coated. In one aspect of the invention, the weight of the coating (e.g., Opadry Pink) is from 0.1% to 10% of the total weight of the unit dosage form. In one aspect, the weight of the coating is from 0.1% to 8% of the total weight of the unit dosage form. In another aspect of this embodiment, the weight of the coating is from 0.1% to 5% of the total weight of the unit dosage form.

The invention also provides a dosage form having between 320 to 480 mg (R)-2-(2-fluoro-4-biphenylyl)propionic acid where the unit dosage form is no longer than 0.82 inches, no longer than 0.80 inches, no longer than 0.77 inches, no longer than 0.72 inches, or no longer than 0.70 inches. The formulation of this embodiment can be a unit dosage form suited for oral administration (e.g., a tablet). In one aspect of this embodiment, the unit dosage form is no wider than 0.41 inches, no wider than 0.40 inches, no wider than 0.38 inches, or no wider than 0.35 inches. In some aspects of the invention, the total volume of the unit dosage form is less than 0.70 cm³, less than 0.65 cm³, less than 0.60 cm³, less than 0.55 cm³, less than 0.50 cm³, or less than 0.45 cm³.

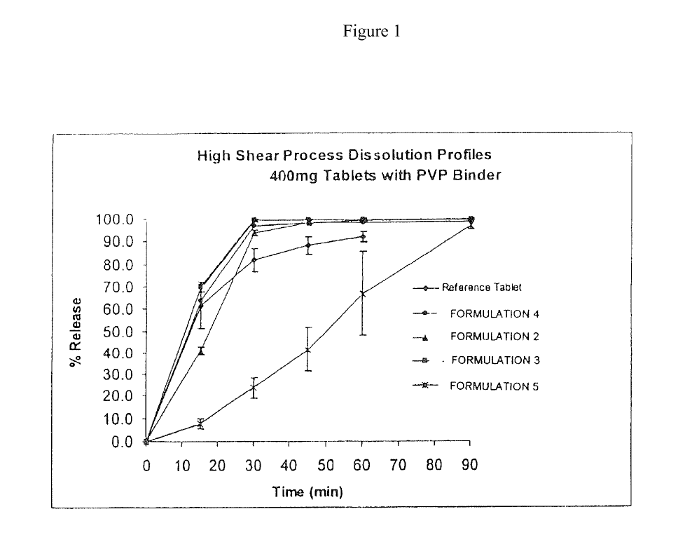

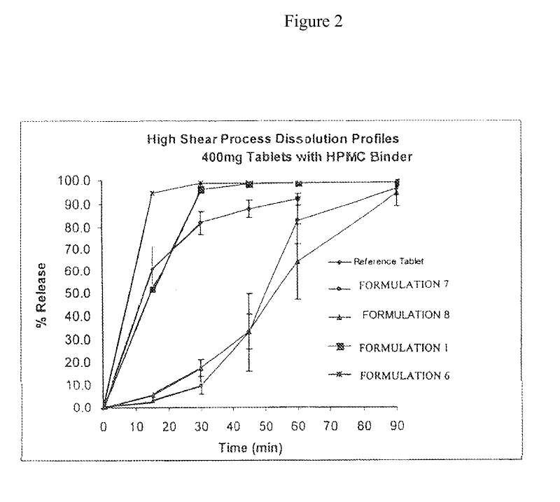

Furthermore, the invention provides a unit dosage form having 55% or more by weight of (R)-2-(2-fluoro-4-biphenylyl)propionic acid that yields a dissolution profile that is substantially similar to one or more of those shown in FIGS. 1 and 2. The unit dosage form of this embodiment is suited for oral administration. For the purpose of comparing dissolution profiles, the method disclosed in Example 3 can be used. In one aspect of this embodiment, the unit dosage form has about 400 mg of (R)-2-(2-fluoro-4-biphenylyl)propionic acid and has a dissolution profile substantially similar to that shown for Formulation 1 in FIG. 2.

The invention also provides a formulation having (R)-2-(2-fluoro-4-biphenylyl)propionic acid, one or more disintegrants, one or more binders, one or more diluents, and if desired, optional ingredients. In one aspect of this embodiment, (R)-2-(2-fluoro-4-biphenylyl)propionic acid is present in an amount of from 55% to 90% by weight, disintegrant from 5% to 45% by weight, binder from 1% to 20% by weight, diluent from 1% to 20% by weight, and any optional ingredients. The unit dosage form of this embodiment is suited for oral administration.

According to one aspect of the invention, the formulation has from 55% to 90% by weight (R)-2-(2-fluoro-4-biphenylyl)propionic acid, 1% to 20% by weight lactose, 1% to 20% by weight hydroxypropyl methylcellulose, 5% to 45% by weight microcrystalline cellulose, and if desired, optional ingredients. According to another aspect of this embodiment, the formulation has from 55% to 85% by weight (R)-2-(2-fluoro-4-biphenylyl)propionic acid, 3% to 17% by weight lactose, 1% to 15% by weight hydroxypropyl methylcellulose, 5% to 25% by weight microcrystalline cellulose, and if desired, optional ingredients. According to yet another aspect of this embodiment, the formulation has from 55% to 80% by weight (R)-2-(2-fluoro-4-biphenylyl)propionic acid, 5% to 15% by weight lactose, 2% to 10% by weight hydroxypropyl methylcellulose, 10% to 20% by weight microcrystalline cellulose, and if desired, optional ingredients. According to another aspect of this embodiment, the formulation has from 60% to 70% by weight (R)-2-(2-fluoro-4-biphenylyl)propionic acid, 8% to 12% by weight lactose, 5% to 8% by weight hydroxypropyl methylcellulose, 12% to 16% by weight microcrystalline cellulose, and if desired, optional ingredients.

The invention further provides an orally available composition having (R)-2-(2-fluoro-4-biphenylyl)propionic acid, lactose, colloidal silicon dioxide, hydroxypropyl methylcellulose, microcrystalline cellulose, croscarmellose sodium, and magnesium stearate. According to a specific aspect of this embodiment, the formulation has from 55% to 90% by weight (R)-2-(2-fluoro-4-biphenylyl)propionic acid, 1% to 20% by weight lactose, 0.1% to 7% by weight colloidal silicon dioxide, 1% to 20% by weight hydroxypropyl methylcellulose, 5% to 45% by weight microcrystalline cellulose, 0.1% to 10% by weight croscarmellose sodium, 0.1% to 10% by weight magnesium stearate, and optional ingredients as desired. According to a more specific aspect of this embodiment, the formulation has from 55% to 85% by weight (R)-2-(2-fluoro-4-biphenylyl)propionic acid, 3% to 17% by weight lactose, 0.1% to 5% by weight colloidal silicon dioxide, 1% to 15% by weight hydroxypropyl methylcellulose, 5% to 25% by weight microcrystalline cellulose, 0.1% to 5% by weight croscarmellose sodium, 0.1% to 5% by weight magnesium stearate, and optional ingredients as desired. In an even more specific aspect of this embodiment, the formulation has from 55% to 80% by weight (R)-2-(2-fluoro-4-biphenylyl)propionic acid, 5% to 15% by weight lactose, 0.1% to 3% by weight colloidal silicon dioxide, 2% to 10% by weight hydroxypropyl methylcellulose, 10% to 20% by weight microcrystalline cellulose, 0.1% to 3% by weight croscarmellose sodium, 0.1% to 3% by weight magnesium stearate, and optional ingredients as desired. According to another specific aspect of this embodiment, the formulation has from 60% to 70% by weight (R)-2-(2-fluoro-4-biphenylyl)propionic acid, 8% to 12% by weight lactose, 0.5% to 2% by weight colloidal silicon dioxide, 5% to 8% by weight hydroxypropyl methylcellulose, 12% to 16% by weight microcrystalline cellulose, 0.2% to 2% by weight croscarmellose sodium, 0.2% to 2% by weight magnesium stearate, and optional ingredients as desired.

In one embodiment, the invention provides a unit dosage form having 55% or more by weight of (R)-2-(2-fluoro-4-biphenylyl)propionic acid where the (R)-2-(2-fluoro-4-biphenylyl)propionic acid is obtained from flurbiprofen. In one aspect of this embodiment, flurbiprofen is prepared from 4-bromo-2-fluorobiphenyl. In another aspect of this embodiment, the (R)-2-(2-fluoro-4-biphenylyl)propionic acid in the unit dosage form is obtained by chiral recrystallization from the racemate.

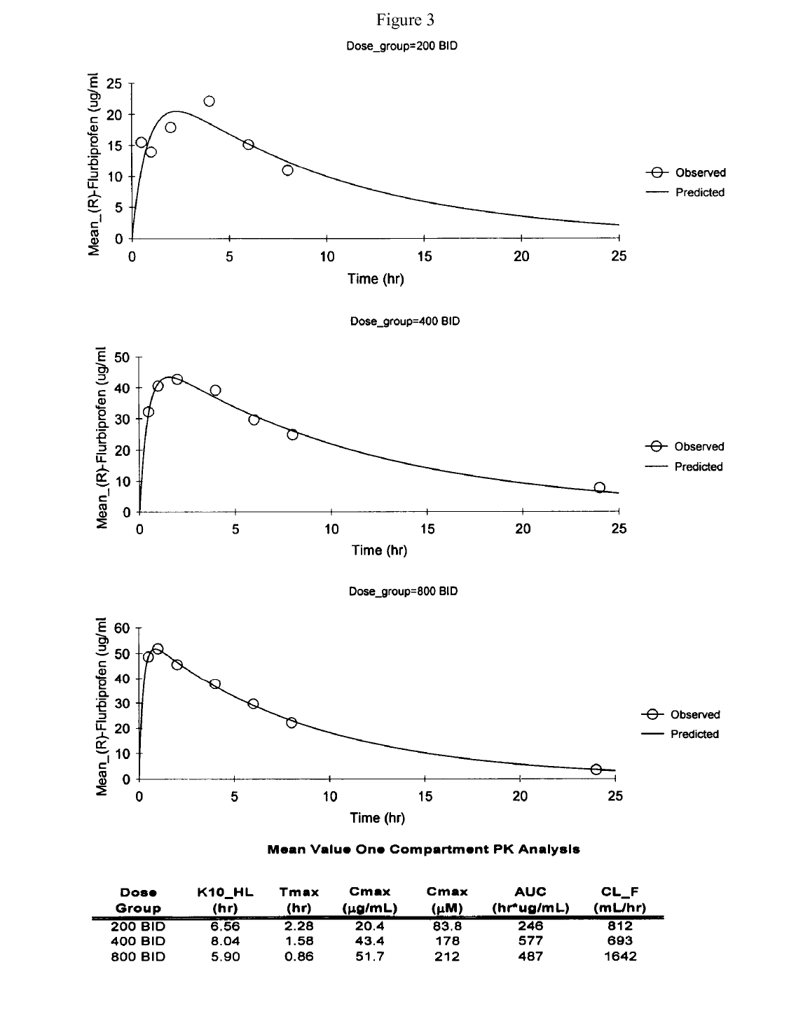

The invention also relates to (R)-2-(2-fluoro-4-biphenylyl)propionic acid unit dosage forms having 55% or more by weight of (R)-2-(2-fluoro-4-biphenylyl)propionic acid that yield a pharmacokinetic profile that is substantially bioequivalent to that shown in FIG. 3. As used herein, substantially bioequivalent refers to Cmax (maximum plasma concentration) and AUC (area under the curve; drug exposure) parameters within 80% to 125% of the reference parameter. The unit dosage form of this embodiment is suited for oral administration (e.g., a tablet). In some aspects of this embodiment, the unit dosage form is a coated tablet.

In one embodiment, oral administration of a single dose (e.g., two tablets each having 400 mg API) of the formulation of the invention to a fasting subject, provides a Cmax of about 25-200 μg per mL per dose, preferably 25-150 μg per mL per dose, and more preferably, between 30-95 μg per mL per dose. In some aspects of the invention, oral administration of a single dose of the formulation of the invention to a fasting subject, provides a Cmax, per dose, of greater than 25 μg per mL, 30 μg per mL, 35 μg per mL, 40 μg per mL, 45 μg per mL, 50 μg per mL, 55 μg per mL, or 60 μg per mL. Administration of a single dose of the compositions of the invention to a fasting subject provides an AUC (area under curve of concentration versus time; total drug exposure) of from about 200 hr·μg/mL to about 600 hr·μg/mL. It is understood by the skilled artisan that the pharmacokinetic parameters can vary substantially depending on the subject (patient taking the drug) and these values are representative of parameters obtained from a group of subjects, rather than one individual. See US Patent Publication No. 20050042284 (U.S. Ser. No. 10/889,971 to Zavitz et. al, filed Jul. 12, 2004) which is hereby incorporated by reference for a description of methods for obtaining these pharmacokinetic parameters.

Desirably, the formulations of the invention are substantially free of (S)-2-(2-fluoro-4-biphenylyl)propionic acid. In one aspect, at least 90% by weight (R)-2-(2-fluoro-4-biphenylyl)propionic acid to 10% by weight or less of (S)-2-(2-fluoro-4-biphenylyl)propionic acid of the total 2-(2-fluoro-4-biphenyl)propionic acid (S+R) is in the pharmaceutical composition. In another aspect, at least 95% by weight (R)-2-(2-fluoro-4-biphenylyl)propionic acid to 5% by weight or less of (S)-2-(2-fluoro-4-biphenylyl)propionic acid of the total 2-(2-fluoro-4-biphenyl)propionic acid (S+R) is in the pharmaceutical composition. In yet another aspect, at least 99% by weight (R)-2-(2-fluoro-4-biphenylyl)propionic acid to 1% by weight or less of (S)-2-(2-fluoro-4-biphenylyl)propionic acid of the total 2-(2-fluoro-4-biphenylyl)propionic acid (S+R) is in the pharmaceutical composition. In yet another aspect, at least 99.9% by weight (R)-2-(2-fluoro-4-biphenylyl)propionic acid to 0.1% by weight or less of (S)-2-(2-fluoro-4-biphenylyl)propionic acid of the total 2-(2-fluoro-4-biphenyl)propionic acid (S+R) is in the pharmaceutical composition. In one aspect, the unit dosage form is a tablet. In another aspect, the unit dosage form is a capsule.

In one specific embodiment of the invention, a tablet unit dosage form is provided having from about 380 mg to 420 mg (R)-2-(2-fluoro-4-biphenylyl)propionic acid, from about 50 mg to 70 mg lactose, from about 3 mg to 7 mg colloidal silicon dioxide, from about 30 mg to 50 mg hydroxypropyl methylcellulose, from about 70 mg to 105 mg microcrystalline cellulose, from about 1 mg to 5 mg croscarmellose sodium, from about 4 mg to 8 mg magnesium stearate, and optional ingredients as desired. According to a more specific aspect of this embodiment, the formulation has from about 385 mg to 415 mg (R)-2-(2-fluoro-4-biphenylyl)propionic acid, from about 55 mg to 65 mg lactose, from about 3.5 mg to 6.5 mg colloidal silicon dioxide, from about 32 mg to 48 mg hydroxypropyl methylcellulose, from about 75 mg to 100 mg microcrystalline cellulose, from about 1.5 mg to 4.5 mg croscarmellose sodium, from about 4.5 mg to 7.5 mg magnesium stearate, and optional ingredients as desired. According to an even more specific aspect of this embodiment, the formulation has from about 390 mg to 410 mg (R)-2-(2-fluoro-4-biphenylyl)propionic acid, from about 56 mg to 64 mg lactose, from about 4.0 mg to 6.5 mg colloidal silicon dioxide, from about 34 mg to 46 mg hydroxypropyl methylcellulose, from about 80 mg to 95 mg microcrystalline cellulose, from about 2.0 mg to 4.0 mg croscarmellose sodium, from about 5.0 mg to 7.0 mg magnesium stearate, and optional ingredients as desired. In an even more specific aspect of this embodiment, the formulation has from about 395 mg to 405 mg (R)-2-(2-fluoro-4-biphenylyl)propionic acid, from about 56 mg to 64 mg lactose, from about 4.0 mg to 6.0 mg colloidal silicon dioxide, from about 34 mg to 46 mg hydroxypropyl methylcellulose, from about 82 mg to 93 mg microcrystalline cellulose, from about 2.0 mg to 4.0 mg croscarmellose sodium, from about 5.0 mg to 7.0 mg magnesium stearate, and optional ingredients as desired.

Definitions

As used herein, the term “(R)-2-(2-fluoro-4-biphenylyl)propionic acid” refers to the free acid form of (R)-2-(2-fluoro-4-biphenylyl)propionic acid and molar equivalents of various salt forms, substantially free of (S)-2-(2-fluoro-4-biphenylyl)propionic acid. When the term “(R)-2-(2-fluoro-4-biphenylyl)propionic acid” is used herein, it is also to be interpreted to include pharmaceutically acceptable salts thereof. In the context of specific amounts and ranges of pharmaceutically acceptable salts, it is to be interpreted as an equivalent molar amount of the free acid. That is to say that if a pharmaceutically acceptable salt is used in the formulation, it should provide the same molar amount of the free acid form as specified in the particular embodiment.

As used herein, the term “dose” or “dosage” refers to the amount of active pharmaceutical ingredient that an individual takes or is administered at one time. For example, an 800 mg (R)-2-(2-fluoro-4-biphenylyl)propionic acid dose refers to, in the case of a twice-daily dosage regimen, a situation where, for example, the individual takes 800 mg (R)-2-(2-fluoro-4-biphenylyl)propionic acid in the morning and 800 mg (R)-2-(2-fluoro-4-biphenylyl)propionic acid in the evening. The 800 mg (R)-2-(2-fluoro-4-biphenylyl)propionic acid dose can be divided into two or more dosage units, e.g., two 400 mg (R)-2-(2-fluoro-4-biphenylyl)propionic acid tablets or two 400 mg (R)-2-(2-fluoro-4-biphenylyl)propionic acid capsules. As used herein, the term “unit dosage form” refers to a physically discrete unit, such as a capsule or tablet suitable as a unitary dosage for a human patient. Each unit contains a predetermined quantity of (R)-2-(2-fluoro-4-biphenylyl)propionic acid that was discovered as a result of this invention to produce the desired pharmacokinetic profile which yields the desired therapeutic effect.

As used herein, the phrase “A dissolution profile substantial similar” refers to one that gives within ±50, 40, 30, 20, 10, or 5% of indicated release of the API when tested according to the procedure set forth in Example 3 at specific time points.

Methods for Preparing Unit Dosage Forms

In general, there are three general methods of tablet preparation: (1) the wet-granulation method; (2) the dry-granulation method; and (3) direct compression. These methods are well known to those skilled in the art. See, Remington's Pharmaceutical Sciences, 16th and 18th Eds., Mack Publishing Co., Easton, Pa. (1980 and 1990). See, also, U.S. Pharmacopeia XXI, U.S. Pharmacopeial Convention, Inc., Rockville, Md. (1985).

In one embodiment, the (R)-2-(2-fluoro-4-biphenylyl)propionic acid tablets can be manufactured using a high shear wet granulation method incorporating pre-blending and pre-milling to reduce the size of the large particles in the drug substance. Once granulated, the material can be dried, milled and blended again. The final powder blend can be compressed into tablets on a high-speed rotary press and the resulting tablets coated in a perforated pan.

Soft gelatin capsules can be prepared in which capsules contain a mixture of the active pharmaceutical ingredient and vegetable oil or non-aqueous, water miscible materials such as, for example, polyethylene glycol and the like. Hard gelatin capsules may contain granules of the active pharmaceutical ingredient in combination with a solid, pulverulent carrier, such as, for example, lactose, saccharose, sorbitol, mannitol, potato starch, corn starch, amylopectin, cellulose derivatives, or gelatin.

Tablets are typically made by molding, by compression or by generally accepted tablet forming methods. Accordingly, compressed tablets are usually prepared by large-scale production methods while molded tablets often involve small-scale operations.

Tablets for oral use are typically prepared in the following manner, although other techniques may be employed. The solid substances are ground or sieved to a desired particle size, and the binding agent is homogenized and suspended in a suitable solvent. The active pharmaceutical ingredient and auxiliary agents are mixed with the binding agent solution. The resulting mixture is moistened to form a uniform suspension. The moistening typically causes the particles to aggregate slightly, and the resulting mass is gently pressed through a stainless steel sieve having a desired size. The layers of the mixture are then dried in controlled drying units for determined length of time to achieve a desired particle size and consistency. The granules of the dried mixture are gently sieved to remove any powder. To this mixture, disintegrating, anti-friction, and anti-adhesive agents are added. Finally, the mixture is pressed into tablets using a machine with the appropriate punches and dies to obtain the desired tablet size. The operating parameters of the machine may be selected by the skilled artisan.

Various tablet formulations may be made in accordance with the present invention. These include tablet dosage forms such as sugar-coated tablets, film-coated tablets, enteric-coated tablets, multiple-compressed tablets, prolonged action tablets and the like. Sugar-coated tablets (SCT) are compressed tablets containing a sugar coating. Such coatings may be colored and are beneficial in covering up drug substances possessing objectionable tastes or odors and in protecting materials sensitive to oxidation. Film-coated tablets (FCT) are compressed tablets that are covered with a thin layer or film of a water-soluble material. A number of polymeric substances with film-forming properties may be used. The film coating imparts the same general characteristics as sugar coating with the added advantage of a greatly reduced time period required for the coating operation. Enteric-coated tablets are also suitable for use in the present invention. Enteric-coated tablets (ECT) are compressed tablets coated with substances that resist dissolution in gastric fluid but disintegrate in the intestine. Enteric coating can be used for tablets containing drug substances that are inactivated or destroyed in the stomach, for those which irritate the mucosa or as a means of delayed release of the medication.

Multiple compressed tablets (MCT) are compressed tablets made by more than one compression cycle, such as layered tablets or press-coated tablets. Layered tablets are prepared by compressing additional tablet granulation on a previously compressed granulation. The operation may be repeated to produce multilayered tablets of two, three or more layers. Typically, special tablet presses are required to make layered tablets. See, for example, U.S. Pat. No. 5,213,738, incorporated herein in its entirety by reference thereto.

Press coated tablets are another form of multiple compressed tablets. Such tablets, also referred to as dry-coated tablets, are prepared by feeding previously compressed tablets into a tableting machine and compressing another granulation layer around the preformed tablets. These tablets have all the advantages of compressed tablets, i.e., slotting, monogramming, speed of disintegration, etc., while retaining the attributes of sugar coated tablets in masking the taste of the drug substance in the core tablet. Press-coated tablets can also be used to separate incompatible drug substances. Further, they can be used to provide an enteric coating to the core tablets. Both types of tablets (i.e., layered tablets and press-coated tablets) may be used, for example, in the design of prolonged-action dosage forms of the present invention.

In practical use, (R)-2-(2-fluoro-4-biphenylyl)propionic acid can be combined as the active pharmaceutical ingredient in intimate admixture with a pharmaceutically acceptable carrier according to conventional pharmaceutical compounding techniques. In preparing the compositions for oral dosage form, any of the usual pharmaceutical media or excipients may be employed. These include, for example, water, glycols, oils, alcohols, flavoring agents, preservatives, coloring agents and the like in the case of oral liquid preparations such as suspensions, elixirs and solutions; or aerosols; or excipients such as starches, sugars, microcrystalline cellulose, diluents, granulating agents, lubricants, binders, disintegrating agents and the like in the case of oral solid preparations such as powders, capsules, caplets, and tablets. Solid oral preparations are generally preferred over liquid ones. Because of their ease of administration, tablets and capsules represent the most advantageous oral dosage unit forms, in which case solid pharmaceutical pharmaceutically acceptable excipients are obviously employed. If desired, tablets may be coated by standard aqueous or nonaqueous techniques. Preferred solid oral preparations are tablets and capsules.

Pharmaceutical stabilizers may be used to stabilize compositions comprising (R)-2-(2-fluoro-4-biphenylyl)propionic acid, or pharmaceutically acceptable salts, solvates, or clathrates thereof. Acceptable stabilizers include, but are not limited to, L-cysteine hydrochloride, glycine hydrochloride, malic acid, sodium metabisulfite, citric acid, tartaric acid, and L-cystine dihydrochloride. See, e.g., U.S. Pat. Nos. 5,731,000; 5,763,493; 5,541,231; and 5,358,970, all of which are incorporated herein by reference.

In general, the compositions are prepared by uniformly and intimately admixing the active pharmaceutical ingredient with a liquid pharmaceutically acceptable carrier or a finely divided solid pharmaceutically acceptable carrier, or both, and then, if necessary, shaping the product into the desired presentation. For example, a tablet may be prepared by compression or molding, optionally with one or more accessory ingredients. Compressed tablets may be prepared by compressing in a suitable machine the active pharmaceutical ingredient in a free-flowing form such as powder or granules, optionally mixed with a binder, lubricant, inert diluent, disintegrating agent, and/or surface active or dispersing agent. Molded tablets may be made by molding in a suitable machine a mixture of the powdered compound moistened with an inert liquid diluent.

The invention relates to the preparation of high drug load formulations having (R)-2-(2-fluoro-4-biphenylyl)propionic acid as the active ingredient. The inventors have discovered formulations of (R)-2-(2-fluoro-4-biphenylyl)propionic acid that allow for the production of (R)-2-(2-fluoro-4-biphenylyl)propionic acid unit dosage forms having 200 mg or more of API, excellent mechanical properties, and a therapeutically desirable pharmacokinetic profile (and dissolution profile). The inventive formulations also allow for the production of tablets having 55% or more active ingredient (by weight). In particular, the invention relates to processes and compositions useful in the preparation of (R)-2-(2-fluoro-4-biphenylyl)propionic acid containing unit dosage forms.

The invention provides compositions and methods useful for preparing unit dosage forms having (R)-2-(2-fluoro-4-biphenylyl)propionic acid as the active pharmaceutical ingredient. According to one embodiment of the invention, the composition is a pre-blend composition having (R)-2-(2-fluoro-4-biphenylyl)propionic acid, one or more diluents, and one or more glidants as ingredients. In some aspects of this embodiment, (R)-2-(2-fluoro-4-biphenylyl)propionic acid is present in the pre-blend composition in amounts from 50-95%, 60-95%, or 70-95% of the total weight of the pre-blend composition. In some aspects of this embodiment, the pre-blend composition has one or more diluents present in amounts from 1-30%, 3-25%, or 5-20% of the total weight of the pre-blend composition. In some aspects of this embodiment, the pre-blend composition has one or more glidants present in amounts from 0.01-5%, 0.1-5%, or 0.1-3% of the total weight of the pre-blend composition. In some aspects of this embodiment optional ingredients are present in amounts from 0-20%, 1-20%, or 1-10% of the total weight of the pre-blend composition. The methods of this embodiment include charging (R)-2-(2-fluoro-4-biphenylyl)propionic acid, the one or more diluents, the one or more glidants and any optional ingredients in a blender followed by blending for a sufficient amount of time to provide a substantially uniform mixture. The blended pre-mill composition can then be used in the next step of the process—milling. Accordingly, the pre-mill composition is then milled through a screen sufficient to reduce the particle size of the pre-mill composition. The milled composition can then be used to form a wet granulation.

The invention provides a wet granulation composition having (R)-2-(2-fluoro-4-biphenylyl)propionic acid, one or more diluents, one or more glidants, one or more binders, one or more wetting agents and optionally, one or more additional ingredients. Furthermore, in one embodiment, the invention provides a method for wet granulation of the wet granulation composition. In one aspect of this embodiment, (R)-2-(2-fluoro-4-biphenylyl)propionic acid is present in the wet granulation composition in amounts from 40-95%, 45-95%, or 50-90% of the total weight of the wet granulation composition. In one aspect of this embodiment, the one or more diluents are present in the wet granulation composition in amounts from 1-30%, 1-20%, or 5-15% of the total weight of the wet granulation composition. In one aspect of this embodiment, the one or more glidants are present in the wet granulation composition in amounts from 0.01-10, 0.01-5%, or 0.1-5% of the total weight of the wet granulation composition. In one aspect of this embodiment, the one or more binders are present in the wet granulation composition in amounts from 1-30%, 1-20%, or 1-15% of the total weight of the wet granulation composition. In one aspect of this embodiment, the one or more wetting agents are present in the wet granulation composition in amounts from 1-40%, 1-25%, or 5-25% of the total weight of the wet granulation composition. In some aspects of this embodiment, optional ingredients are present in amounts from 0-20%, 1-20%, or 1-10% of the total weight of the wet granulation composition. According to this embodiment, the one or more binders, milled composition, and any optional ingredients are charged into a granulator and dry blended for a sufficient amount of time followed by granulation with the wetting agent for a sufficient amount of time. The wet granulation is then milled through a screen. The wet granulation is then dried by a method appropriate for removing the wetting agent to yield a dried granulation.

In another embodiment, the invention provides a pre-tableting composition having a dried granulation component and one or more disintegrants. Thus, this embodiment provides a pre-tableting composition having (R)-2-(2-fluoro-4-biphenylyl)propionic acid, one or more diluents, one or more binders, one or more glidants, one or more disintegrants, and optionally, one or more optional ingredients, and methods of preparing the pre-tableting composition. In one aspect of this embodiment, (R)-2-(2-fluoro-4-biphenylyl)propionic acid is present in the pre-tableting composition in amounts from 50-95%, 55-90%, or 55-85% of the total weight of the intra-granular composition. In one aspect of this embodiment, the one or more diluents are present in the pre-tableting composition in amounts from 1-30%, 1-20%, or 5-15% of the total weight of the pre-tableting composition. In one aspect of this embodiment, the one or more glidants are present in the pre-tableting composition in amounts from 0.01-10, 0.01-5%, or 0.1-5% of the total weight of the pre-tableting composition. In one aspect of this embodiment, the one or more binders are present in the pre-tableting composition in amounts from 1-30%, 1-20%, or 1-15% of the total weight of the pre-tableting composition. In one aspect of the embodiment, the one or more disintegrants are present in amounts from 1-40%, 5-25% or 5-20% of the total weight of the pre-tableting composition. In one aspect of this embodiment, one or more optional ingredients are present in the pre-tableting composition in amounts from 1-20%, 1-25%, or 5-25% of the total weight of the pre-tableting composition.

According to the method of this embodiment, the pre-tableting composition is made by blending (R)-2-(2-fluoro-4-biphenylyl)propionic acid, one or more binders, one or more glidants, and any optional ingredients in a blender for a sufficient amount of time followed by milling of the resultant blend through a screen of sufficient mess size to decrease the size of API containing particles. The milled composition is then placed or discharged into a high shear granulator with one or more disintegrants, and any optional ingredients where these components are dry blended for a sufficient amount of time to provide a uniform mixture. Next the dry blended material is granulated with purified water (5-30% of the dry weight of the material, or 5-25% of the dry weight of the material, or 10-22% of the dry weight of the material), for a sufficient amount of time to yield a wet granulation. Next, the wet granulation is milled through a screen of appropriate size followed by drying of the milled wet granulation. Lastly the dried milled wet granulation is milled through a screen of appropriate size to yield a dry granulation (pre-tableting composition).

In another embodiment, the invention provides method for preparing a tableting composition having the pre-tableting composition and one or more lubricants. Thus, this embodiment relates to formulations having (R)-2-(2-fluoro-4-biphenylyl)propionic acid as an API, one ore more diluents, one or more binders, one or more glidants, one or more disintegrants, and one or more lubricants, and methods of preparing such compositions. The composition of this embodiment is suited for compression tableting. In one aspect of this embodiment, (R)-2-(2-fluoro-4-biphenylyl)propionic acid is present in the pre-tableting composition in amounts from 50-95%, 55-90%, or 55-85% of the total weight of the tableting composition. In one aspect of this embodiment, the one or more diluents are present in the tableting composition in amounts from 1-30%, 1-20%, or 5-15% of the total weight of the tableting composition. In one aspect of this embodiment, the one or more binders are present in the tableting composition in amounts from 1-30%, 1-20%, or 1-15% of the total weight of the tableting composition. In one aspect of this embodiment, the one or more glidants are present in the tableting composition in amounts from 0.01-10, 0.01-5%, or 0.1-5% of the total weight of the tableting composition. In one aspect of this embodiment, the one or more disintegrants are present in the tableting composition in amounts from 1-40%, 5-25%, or 5-20% of the total weight of the tableting composition. The lubricant is present in an amount sufficient to allow ejection of the tablet cleanly from the die with minimal stress to the tablet. In one aspect of this embodiment, the one or more lubricants are present in amounts from 0.01-10%, 0.1-10%, or 0.1-5% of the total weight of the composition of this embodiment. According to the method of this embodiment, the composition is prepared by charging the one or more lubricants into the diffusion blender with the other components (e.g., those in embodiment four) and blending for an amount of time sufficient to provide a uniform mixture. The composition prepared according to this embodiment can then be compressed into tablets with an appropriate press. The composition is sufficiently compressed to yield a tablet that, when coated, yields an immediate release dissolution profile similar to one or more of those shown in FIGS. 1 and 2.

In another embodiment, the invention relates to preparing a coated tablet having (R)-2-(2-fluoro-4-biphenylyl)propionic acid as an API. The tablet (i.e., those prepared according to the fourth embodiment) is coated with a coating sufficient to yield an immediate release dissolution profile of the coated tablet unit dosage form and/or to impart sufficient stability to the unit dosage form. According to this embodiment, a film coating suspension is prepared with a suitable coating agent and water. The film coating suspension can then used to coat the tablets in, e.g., a perforated coating pan to yield a coated tablet. In some aspects of this embodiment, the coating represents from 0.1-15%, 0.1-10%, or 1-7% of the total weight of the tablet.

In another embodiment, the invention provides an (R)-2-(2-fluoro-4-biphenylyl)propionic acid tablet unit dosage form produced according to the methods of the invention that yields a dissolution profile similar to one or more of those shown in FIGS. 1 and 2 and/or a pharmacokinetic profile bioequivalent to one or more of those shown in FIG. 3.

In a specific embodiment, (R)-2-(2-fluoro-4-biphenylyl)propionic acid tablets can be manufactured using a high shear granulation method, incorporating pre-blending and pre-milling to reduce the size of the large particles in the drug substance. Once granulated, the material was dried, milled and blended again. The final powder blend was compressed into tablets on a high-speed rotary press and the resulting tablets were coated in a perforated pan. Bulk coated tablets were bulk-packed for shipping prior to clinical packaging.

In some aspects of the above-described embodiments, the unit dosage form of the invention can be manufactured using a high shear granulation process in which (R)-2-(2-fluoro-4-biphenylyl)propionic acid is pre-blended and pre-milled with one or more binders such as anhydrous lactose and one or more glidants such as colloidal silicon dioxide. The pre-blend can be processed in a drum blender followed by milling to decrease the median particle size of the large particles of the (R)-2-(2-fluoro-4-biphenylyl)propionic acid pre-blend prior to high shear granulation. Once granulated, the pre-blend can be dried, milled, blended, compressed on a high-speed rotary press and coated in a perforated pan.

In one aspect, the invention provides a method of manufacturing a tablet unit dosage form having (R)-2-(2-fluoro-4-biphenylyl)propionic acid as an active pharmaceutical ingredient comprising:

(a) charging the lactose, (R)-2-(2-fluoro-4-biphenylyl)propionic acid drug substance, colloidal silicon dioxide, and hydroxypropyl methylcellulose into the high shear granulator;

(b) blending (e.g., dry blending) the lactose, (R)-2-(2-fluoro-4-biphenylyl)propionic acid drug substance, colloidal silicon dioxide, and hydroxypropyl methylcellulose in the high shear granulator;

(c) granulating the material using purified water;

(d) milling the wet granulation through an appropriate size screen;

(e) drying the milled granulation;

(f) milling the dried granulation through an appropriate size screen;

(g) charging dried granulation along with microcrystalline cellulose, croscarmellose sodium, and colloidal silicon dioxide into a diffusion blender and blending the material for an appropriate amount of time;

(h) charging the magnesium stearate into the diffusion blender and blending for an appropriate amount of time;

(i) compressing the blended powders on a high-speed rotary press; and

(j) film coating the tablets.

Inactive Pharmaceutical Ingredients

The formulations and unit dosage forms of the invention can have a number of different ingredients. Depending on the dosage strength, a unit dosage form has an amount of active pharmaceutical ingredient (API) sufficient for achieving a therapeutic effect in a target population. Additionally “inactive pharmaceutical ingredients” need to be present to achieve a therapeutically effective release of the API. Thus the amount and type of inactive ingredients help achieve a therapeutically effective release of the therapeutic agent. In one aspect of the invention, a tablet unit dosage form is provided having the following inactive ingredients: one or more disintegrants in an amount sufficient to facilitate break-up (disintegration) of the tablet after administration (e.g., provide an immediate release dissolution profile), one or more binders in an amount sufficient to impart adequate cohesiveness to the tablet and/or provide adequate free flowing qualities by formulation of granules of desired size/hardness, one or more diluents in an amount sufficient to impart satisfactory compression characteristics, one or more lubricants in an amount sufficient to provide an adequate flow rate of the granulation and/or prevent adhesion of the material to the die/punch, reduce interparticle friction, and/or facilitate ejection from the die, and if desired, optional ingredients.

The disintegration rate, and often the dissolution rate of a compacted solid pharmaceutical formulation in an aqueous environment (e.g., the patient's stomach) may be increased by the addition of a disintegrant to the formulation. Disintegrants include, but are not limited to, alginic acid, carboxymethylcellulose calcium, carboxymethylcellulose sodium (e.g., Ac-Di-Sol® Primellose®.), colloidal silicon dioxide, croscarmellose sodium, crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®) and starch.

Solid pharmaceutical formulations that are compacted into a dosage form, such as a tablet, may include excipients whose functions include helping to bind the active pharmaceutical ingredient and other excipients together after compression. Binders for solid pharmaceutical formulations include, but are not limited to, acacia, alginic acid, carbomer (e.g., carbopol), carboxymethylcellulose sodium, dextrin, ethyl cellulose, gelatin, guar gum, hydrogenated vegetable oil, hydroxyethyl cellulose, hydroxypropyl cellulose (e.g., Klucel®), hydroxypropyl methylcellulose (e.g., Methocel®), lactose, liquid glucose, magnesium aluminum silicate, maltodextrin, methylcellulose, polymethacrylates, povidone (e.g., Kollidon®, Plasdone®), pregelatinized starch, sodium alginate and starch. Glidants can be added to improve the flowability of a non-compacted solid formulation and to improve the accuracy of dosing. Excipients that may function as glidants include, but are not limited to, colloidal silicon dioxide, magnesium trisilicate, powdered cellulose, starch, talc and tribasic calcium phosphate.

When a dosage form such as a tablet is made by the compaction of a powdered formulation, the formulation is subjected to pressure from a punch and dye. Some excipients and active pharmaceutical ingredients have a tendency to adhere to the surfaces of the punch and dye, which can cause the product to have pitting and other surface irregularities. A lubricant can be added to the formulation to reduce adhesion and ease the release of the product from the dye. Lubricants include, but are not limited to, magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Examples of diluents include, but are not limited to, calcium carbonate, calcium phosphate, calcium sulfate, cellulose, cellulose acetate, compressible sugar, confectioner's sugar, dextrates, dextrin, dextrose, ethyl cellulose, fructose, fumaric acid, glyceryl palmitostearate, hydrogenated vegetable oil, kaolin, lactitol, lactose, magnesium carbonate, magnesium oxide, maltodextrin, maltose, mannitol, medium chaim glyceride, microcrystalline cellulose, polydextrose, polymethylacrylates, simethicone, sodium alginate, sodium chloride, sorbitol, starch, pregelantized starch, sterilizable maize, sucrose, sugar spheres, talc, tragacanth, trehalose, and xylitol.

Examples of disintegrants include, but are not limited to, alginic acid, calcium phosphate, carboxymethyl cellulose calcium, carboxymethyl cellulose sodium, powdered cellulose, chitosan, crospovidone, docusate sodium, guar gum, hydroxylpropyl cellulose, magnesium aluminum silicate, methylcellulose, poidone, sodium alginate, sodium starch glycolate, starch, and pregelantinized starch.

Example of binders (binding agents) include, but are not limited to, acacia, alginic acid, carbomers, carboxymethyl cellulose sodium, carrageenan, cellulose acetate phthalate, ceratonia, chitosan, confectioners sugar, cottonseed oil, dextrates, dextrin, dextrose, ethylcellulose, gelatin, glucose, glyceryl behenate, guar gum, hydrogenated vegetable oil, hydroxyethyl cellulose, hydroxyethylmethyl cellulose, hydroxylpropyl cellulose, hypromellose, magnesium aluminum silicate, maltodextrin, maltodextrin, maltose, methylcellulose, microcrystalline cellulose, poloxamer, polydextrose, polyethylene oxide, polymethyl acrylates, povidone, sodium alginate, starch, pregelantized starch, stearic acid, sucrose, sunflower oil, and zein.

Examples of lubricants include, but are not limited to, calcium stearate, glycerin monostearate, glyceryl behenate, glyceryl palmitostearate, hydrogenated castor oil, hydrogenated vegetable oil, light mineral oil, magnesium lauryl sulfate, magnesium stearate, medium chain triglycerides, mineral oil, poloxamer, polyethylene glycol, sodium benzoate, sodium chloride, sodium lauryl sulfate, sodium stearyl fumarate, stearic acid, talc, and zinc stearate.

Examples of glidants include, but are not limited to, calcium phosphate, calcium silicate, cellulose powdered, colloidal silicon dioxide, magnesium silicate, magnesium trisilicate, silicon dioxide, starch, and talc.

Examples of suitable pharmaceutically acceptable salts the API include, but are not limited to, aluminum, calcium, lithium, magnesium, potassium, sodium and zinc. In addition, organic salts may also be used including, but not limited to salts of lysine, N,N′-dibenzylethylenediamine, chloroprocaine, choline, diethanolamine, ethylenediamine, meglumine (N-methylglucamine), procaine and tris.

Optional ingredients in the formulations of the invention include, but are not limited to, flavors, coloring agents, and stabilizers.

Flavoring agents and flavor enhancers make the dosage form more palatable to the patient. Common flavoring agents and flavor enhancers for pharmaceutical products that may be included in the formulation of the present invention include, but are not limited to, maltol, vanillin, ethyl vanillin, menthol, citric acid, fumaric acid, ethyl maltol and tartaric acid.

Solid and liquid formulations may also be dyed using any pharmaceutically acceptable colorant to improve their appearance and/or facilitate patient identification of the product and unit dosage level.

In one embodiment, the tablet unit dosage form has a hardness of about 5 kp (kilopond) or more, about 7 kp or more, about 9 kp or more, about 11 kp or more, and about 13 kp or more to avoid excessive friability, and a hardness of about 20 kp or less, about 19 kp or less, about 18 kp or less, about 17 kp or less, and about 16 kp or less, is desirable to avoid subsequent difficulty in hydrating the tablet when exposed to gastric fluid. In some aspects of this embodiment, the hardness of the tablet unit dosage form is from 9 kp to 18 kp, 11 kp to 17 kp, and 13 kp to 17 kp. When hardness is in an acceptable range, tablet friability is typically less than about 1.0%, preferably less than about 0.8% and more preferably less than about 0.5%, in a standard test. While the skilled artisan recognizes that there are numerous techniques available for determining hardness, for purposes of comparison, the method used to determine tablet hardness of the unit dosage forms of the invention (as described in Example 6) should be used. Some issues that may cause variations in tablet hardness are inconsistent tablet weight, particle size variations, poor powder compressibility, and insufficient binder level.

One problem encountered with tablet unit dosage forms is that they can often exhibit high friability. Friability is a physical parameter of a solid dosage form that relates to the tablets ability to withstand physical perturbations. Friability is the tendency of a tablet to crumble, chip or break. Dosage forms having a high friability will rapidly dissolve or disintegrate. An optimum unit dosage form will rapidly dissolve or disintegrate and have a low level of friability. The present invention provides this combination of desirable traits in a high drug load formulation. Specifically, the (R)-2-(2-fluoro-4-biphenylyl)propionic acid dosage forms of the invention have excellent dissolution profiles and desirable friabilities. The tablets of the invention have a friability of less than about 1%, meaning that the tablets meet the United States Pharmacopeia standard for tablet friability (which requires a friability of less than 1%). While the skilled artisan recognizes that there are numerous techniques available for determining tablet friability, for purposes of comparison, the method used to determine the friability of the unit dosage forms of the invention (as described in Example 7) should be used. Friable tablets can be caused by low moisture content, insufficient binder, tablet configuration (e.g., sharp versus beveled edges).

In some aspects, the tablet unit dosage forms of the invention have a friability of less than about 1%, less than about 0.9%, less than about 0.8%, less than about 0.7%, less than about 0.6%, less than about 0.5%, and less than about 0.4% (all at 100 rev).

Poor disintegration can come from tablets which are compressed too hard, insufficient disintegrant levels, or too much binder.

In some aspects of the invention, the total volume of the unit dosage form is less than 0.7 cm³, less than 0.65 cm³, less than 0.60 cm³, less than 0.55 cm³, less than 0.50 cm³, or less than 0.45 cm³.

The present invention is illustrated below by reference to the following examples which set forth particularly preferred embodiments. However, it should be noted that these embodiments are illustrative and are not to be construed as limiting the invention in any way.

### EXAMPLES

### Example 1

### Components of an (R)-2-(2-fluoro-4-biphenylyl)propionic Acid Containing Tablet Formulation

The components of this tablet formulation are given in table 1 below. The quantitative composition in both the batch preparation and the individual tablets are given in Table 2 while an exemplary method of preparing the tablets is given in Example 2.

TABLE 1
Components of 400 mg Tablets

| Component | Specification/grade |
|---|---|
| Drug substance - (R)-2-(2-fluoro-4- | Manufacturer's |
| biphenylyl)propionic acid | specification |
| Lactose, anhydrous¹ | EP, USP |
| Colloidal silicon dioxide (Cab-O-Sil M5P) | EP, USP |
| Hydroxypropyl methylcellulose E-5 | EP, USP |
| Microcrystalline cellulose (Avicel PH 302) | EP, USP |
| Croscarmellose sodium Type A (Ac-Di-Sol) | EP, USP |
| Magnesium stearate, non-bovine¹ | EP, USP |
| Water, purified | EP, USP |
| Opadry Pink 03K94003 | In-house specification² |

TABLE 2
Quantitative Composition of (R)-2-(2-fluoro-4-biphenylyl)propionic acid
400 mg Tablets

| | | Representative |
|---|---|---|
| | Weight | batch |
| Component | (mg/tablet) | (grams/batch) |
| Drug Substance: (R)-2-(2-fluoro-4- | 400.00 | 300,000 |
| biphenylyl)propionic acid | | |
| Lactose, anhydrous | 59.60 | 44,700 |
| Colloidal silicon dioxide (Cab-O-Sil | 2.70 | 2,025 |
| M5P) | | |
| Hydroxypropyl methylcellulose E-5 | 39.00 | 29,250 |
| Water, purified | Essentially | 70,200¹ |
| | removed | |
| | during drying | |
| Total (R)-2-(2-fluoro-4- | | 375,975 |
| biphenylyl)propionic acid | | |
| granulation: | | |
| (R)-2-(2-fluoro-4- | | 375,975 |
| biphenylyl)propionic acid Granulation | | |
| Microcrystalline cellulose (Avicel PH | 87.00 | 65,250 |
| 302) | | |
| Croscarmellose sodium (Ac-Di-Sol) | 3.00 | 2,250 |
| Colloidal silicon dioxide (Cab-O-Sil | 2.70 | 2,025 |
| M5P) | | |
| Magnesium stearate, non-bovine | 6.00 | 4,500 |
| Total (R)-2-(2-fluoro-4- | | 450,000 |
| biphenylyl)propionic acid blend for | | |
| compression: | | |
| (R)-2-(2-fluoro-4- | | 450,000 |
| biphenylyl)propionic acid tablet core | | |
| Opadry Pink 03K94003 | 18.56 | 13,920 |
| Water, purified | Essentially | 102,075 |
| | removed | |
| | during drying | |
| Total: | 618.6 | 463,920 |

FORMULATION 1 BLEND PROPERTIES

| | | |
|---|---|---|
| Bulk Density | 0.51 | g/ml |
| Flow Rate Index: | 4.664 | kg/sec |
| Tap Density | 0.62 | g/ml |

Sieve Analysis

| | |
|---|---|
| Mesh Size/% Retained | Mesh Size/% Retained |

| | | | |
|---|---|---|---|
| 40 mesh: | 22% | 80 mesh: | 45% |
| 100 mesh: | 6% | 140 mesh: | 8% |
| 200 mesh: | 6% | 325 mesh: | 7% |
| Pan: | 6% | | |

FORMULATION 1 TABLET PHYSICAL PROPERTIES

| | |
|---|---|
| Weight Variation | 0.598 g avg. (1.11% RSD) |
| Hardness | 15.2 kp avg. (3.8% RSD) |
| Thickness | 5.30 avg. (0.54% RSD) |
| Disintegration (min:sec) | 22:55, 24:15, 26:30 |
| Friability: | 100 rev. 0.28% 400 rev. 0.82% |

FORMULATION 1 COATED TABLET DISSOLUTION

| | |
|---|---|
| Actual Film Coat | 2.2% |
| 15 min: 51.9% (8.0) | 30 min.: 96.1% (1.2) |
| 45 min.: 98.5% (1.5) | 60 min.: 99.0% (1.4) |
| 90 min.: 99.4% (1.2) | |

The unit dosage form of Example 1 is one preferred unit dosage form of the invention. Thus the unit dosage form can e.g., have at 15 min greater than 50% release of API, at 30 min greater than 60% release of API, at 45 min greater than or equal to 80% release of API, at 60 min great than or equal to 80% release of API, and at 90 min greater than or equal to 80% release of API.

### Example 2

### Process for Preparing (R)-2-(2-fluoro-4-biphenylyl)propionic Acid Containing Tablet Formulations

The tablet unit dosage form in Example 1 can be manufactured according to the following protocol.

The manufacturing procedure was a high shear granulation process incorporating pre-blending and pre-milling to reduce the size of the large particles in the drug substance. Once granulated, the material was dried, milled and blended again. The final powder blend was compressed into tablets on a high-speed rotary press and the resulting tablets were coated in a perforated pan. The outline of the manufacturing is provided below:

1. Charge the lactose anhydrous, (R)-2-(2-fluoro-4-biphenylyl)propionic acid drug substance, and colloidal silicon dioxide into a drum-type blender.

2. Blend components together for a sufficient amount of time (e.g., 5 min) and discharge into a mill (e.g., Comil U20 or equivalent).

3. Mill through a sufficient size screen (e.g., 0.018″) to decrease the particle size of the large particles in the drug substance. Discharge into a high shear granulator (e.g., Fielder PMA300 (Eastleigh, Hampshire, United Kingdom) or equivalent).

4. Charge the hydroxypropyl methylcellulose into the high shear granulator and dry blend (approximately 3 min).

5. Granulate material using purified water (e.g., 14.5% to 18.9% of the dry weight materials; granulated on Setting 1 for approximately 10 min).

6. Mill the wet granulation through an appropriate size screen (e.g., Comil U20 or equivalent; 0.250″ screen).

7. Dry the milled granulation (e.g., Aeromatic T5 fluid bed (Eastleigh, Hampshire, United Kingdom) or equivalent; ca. 70° C. inlet, ca. 30° C. outlet; dry to LOD<2.0%; ca. 20-25 min).

8. Mill the dried granulation through an appropriate size screen (e.g., Comil U20 (available from Quadro, Waterloo, Ontario, Canada) or equivalent; 0.055″ screen).

Note: Steps 1-8 may be performed as sub-lot granulations to enable adjustment of batch size.

9. Charge dried granulation along with microcrystalline cellulose, croscarmellose sodium, and colloidal silicon dioxide into a diffusion blender (e.g., Bohle PM1000 or equivalent). Blend the material for an appropriate amount of time (e.g., 25 min at 6 rpm).

10. Charge the magnesium stearate into the diffusion blender. Blend for an appropriate amount of time (e.g., 5 min at 6 rpm).

11. Compress the blended powders on a high-speed rotary press into 600 mg (total tablet weight) modified oval tablets debossed with MY4.

12. Prepare the film-coating suspension by mixing Opadry Pink into purified water for a 12% by weight solids concentration.

13. Film coat tablets with Opadry Pink in a perforated coating pan (e.g., Lodige LHC130 Hi-Coater) to a theoretical weight gain of approximately 3%.

Note: Steps 12-13 may be performed as sub-lot coatings, in which case step 14 (consolidation of sub-lots) is required.

14. Consolidate sub-lots as necessary.

### Example 3

### Dissolution

Dissolution testing of (R)-2-(2-fluoro-4-biphenylyl)propionic acid 400 mg tablets is performed in 900 mL of pH 7.2 potassium phosphate buffer at 37° C. using USP Apparatus 2 (paddles) at a rotation speed of 75 rpm. At the appropriate time intervals, an aliquot is withdrawn and the amount of dissolved (R)-2-(2-fluoro-4-biphenylyl)propionic acid is determined by isocratic HPLC analysis. The HPLC system consists of a Zorbax 5 μm, SB C18, 250 mm×4.6 mm i.d. column with mobile phase of pH 3.0 potassium phosphate buffer:acetonitrile (30:70). The flow rate is set at 1.0 mL/min and detection is by UV absorption at 247 nm. See US Patent Publication No. 2005042284 for a description of the reference tablets used in obtaining the dissolution profiles in FIG. 1 and FIG. 2.

### Example 4

### Content Uniformity

Content uniformity of the (R)-2-(2-fluoro-4-biphenylyl)propionic acid tablets is determined by reversed-phase HPLC. (R)-2-(2-fluoro-4-biphenylyl)propionic acid is extracted from ten individual tablets by shaking in methanol for thirty minutes. Aliquots of the resulting solutions are then diluted with water:acetonitrile (55:45) and filtered through 0.45 μm nylon Acrodisc syringe filters. The solutions are then injected on to an HPLC system utilizing a Waters Nova-Pak C18, 150×3.9 mm, 4 μm column maintained at 30° C. The injection volume is 10 μL and the mobile phase consists of water:acetonitrile:glacial acetic acid (55:40:5). The flow rate is 1.5 mL/min and detection is by UV absorption at 254 nm.

### Example 5

### Disintegration Rates

The disintegration times of the unit dosage forms of the invention were measured by using USP XXIV disintegration apparatus (See page 1941 of the United States Pharmacopeia XXIV, United States Pharmacopeia commission, Rockville, Md., USA).

### Example 6

### Tablet Hardness

A Key International (Cottage Grove, Oreg.) hardness tester was used to measure tablet hardness.

### Example 7

### Tablet Friability

A Vanderkamp Friabulator Tablet Tester (Vankel Industries, Inc., Cary, N.C.)) was used to measure the friability of the unit dosage forms of the invention (Journal of American Pharmaceutical Assoc. vol. XLV, No. 2 (February 1956).

### Example 8

### Flow Rate

The flow rate index was obtained using a J.R. Johanson Flow Indicizer (J.R. Johanson, Inc. San Luis Obispo, Calif.) which estimates the flow rate, feed and bin density, and the Spring back index based on set bin parameters.

### Example 5

### Formulations

The following formulations exemplify the formulations and unit dosage forms are those used to determine the dissolution profiles shown in FIGS. 1 and 2. A process that can be used for preparing such tablets is disclosed in Example 2.

### Formulation 2

Formulation 2 has a high shear granulation with 1.5% by weight PVP intra-granular and 0.5% by weight AcDiSol extra-granular. The formulation has the following components:

INTRA-GRANULAR

| Component | mg/tablet | W/W % |
|---|---|---|
| (R)-2-(2-fluoro-4- | 400.00 | 66.667 |
| biphenylyl)propionic acid | | |
| drug substance | | |
| Lactose, Anhydrous | 77.60 | 12.933 |
| Cab-O-Sil M5P | 2.70 | 0.45 |
| Povidone, K29/32 (PVP) | 9.00 | 1.50 |
| Purified Water | — | — |
| Total of Intra-Granular | 489.30 | 81.55 |

EXTRA-GRANULAR

| Component | mg/tablet | W/W % |
|---|---|---|
| Avicel ® PH 302 | 99.00 | 16.50 |
| Ac-Di-Sol ® | 3.00 | 0.50 |
| Cab-O-Sil M5P | 2.70 | 0.45 |
| Magnesium Stearate NF | 6.00 | 1.00 |
| Total | 600.00 | 100.00 |

FORMULATION 2 BLEND PROPERTIES

| | | |
|---|---|---|
| Bulk Density: | 0.56 | g/ml |
| Flow Rate Index: | 0.913 | kg/sec |
| Tap Density: | 0.73 | g/ml |

Sieve Analysis

| | |
|---|---|
| Mesh Size/% Retained | Mesh Size/% Retained |

| | | | |
|---|---|---|---|
| 40 mesh: | 23.5% | 80 mesh: | 35.7% |
| 100 mesh: | 6.1% | 140 mesh: | 8.6% |
| 200 mesh: | 6.8% | 325 mesh: | 8.7% |
| Pan: | 10.6% | | |

FORMULATION 2 TABLET PROPERTIES

| | |
|---|---|
| Weight Variation: | 0.600 g avg. (0.67% RSD) |
| Hardness: | 15.2 kp avg. (7.0% RSD) |
| Thickness: | 5.38 avg. (0.53% RSD) |
| Disintegration (min:sec): | 23:07, 24:40, 25:19 |
| Friability: | 100 rev. 0.26% 400 rev. 0.87% |

FORMULATION 2 COATED TABLET DISSOLUTION

| | |
|---|---|
| Actual Film Coat | 2.3% |
| 15 min: 41.0% (4.3) | 30 min.: 93.8% (1.5) |
| 45 min.: 98.3% (0.7) | 60 min.: 99.2% (0.6) |
| 90 min.: 99.5% (0.6) | |

### Formulation 3

Formulation 3 has a high Shear Granulation with 1.5% by weight PVP, and 1.5% by weight AcDiSol, both intra-granular, with 5% by weight StaRx 1500 extra-granular. Formulation 3 has the following components:

INTRA-GRANULAR

| Component | mg/tablet | W/W % |
|---|---|---|
| (R)-2-(2-fluoro-4- | 400.00 | 66.667 |
| biphenylyl)propionic acid | | |
| Drug Substance | | |
| Lactose, Anhydrous | 59.60 | 9.933 |
| Cab-O-Sil M5P | 2.70 | 0.45 |
| Ac-Di-Sol ® | 9.00 | 1.50 |
| Povidone, K29/32 (PVP) | 9.00 | 1.50 |
| Purified Water | — | — |
| Total of Intra-Granular | 480.30 | 80.55 |

EXTRA-GRANULAR

| Component | mg/tablet | W/W % |
|---|---|---|
| Avicel ® PH 302 | 81.00 | 13.50 |
| Pregelatinized Starch | 30.00 | 5.00 |
| StaRx 1500 | | |
| Cab-O-Sil M5P | 2.70 | 0.45 |
| Magnesium Stearate NF | 6.00 | 1.00 |
| Total | 600.00 | 100.00 |

FORMULATION 3 BLEND PROPERTIES

| | | |
|---|---|---|
| Bulk Density: | 0.50 | g/ml |
| Flow Rate Index: | 2.619 | kg/sec |
| Tap Density: | 0.65 | g/ml |

Sieve Analysis

| | |
|---|---|
| Mesh Size/% Retained | Mesh Size/% Retained |

| | | | |
|---|---|---|---|
| 40 mesh: | 5.4% | 80 mesh: | 42.5% |
| 100 mesh: | 8.0% | 140 mesh: | 14.4% |
| 200 mesh: | 11.9% | 325 mesh: | 13.0% |
| Pan: | 10.6% | | |

FORMULATION 3 TABLET PROPERTIES

| | |
|---|---|
| Weight Variation: | 0.604 g avg. (0.68% RSD) |
| Hardness: | 14.4 kp avg. (5.2% RSD) |
| Thickness: | 5.41 avg. (0.34% RSD) |
| Disintegration (min:sec): | 11:45, 12:00, 12:29 |
| Friability: | 100 rev. 0.27% 400 rev. 0.95% |

FORMULATION 3 COATED TABLET DISSOLUTION

| | | | |
|---|---|---|---|
| Actual Film Coat | 2.4% | | |
| 15 min: | 70.0% (3.2) | 30 min: | 99.5% (0.7) |
| 45 min: | 99.7% (0.5) | 60 min: | 99.7% (0.7) |
| 90 min: | 99.9% (0.5) | | |

### Formulation 4

Formulation 4 has a high shear granulation with 1.5% by weight PVP binder and 1.5% by weight Ac-Di-Sol disintegrant intra-granular. Formulation 4 has the following components:

INTRA-GRANULAR

| Component | mg/tablet | W/W % |
|---|---|---|
| (R)-2-(2-fluoro-4- | 400.00 | 66.667 |
| biphenylyl)propionic acid | | |
| drug substance | | |
| Lactose, Anhydrous | 74.60 | 12.433 |
| Cab-O-Sil M5P | 2.70 | 0.45 |
| Ac-Di-Sol ® | 9.00 | 1.50 |
| Povidone, USP K29/32 (PVP) | 9.00 | 1.50 |
| Purified Water | — | — |
| Total of Intra-Granular | 495.30 | 82.55 |

EXTRA-GRANULAR

| Component | mg/tablet | W/W % |
|---|---|---|
| Avicel ® PH 302 | 96.00 | 16.00 |
| Cab-O-Sil M5P | 2.70 | 0.45 |
| Magnesium Stearate NF | 6.00 | 1.00 |
| Total | 600.00 | 100.00 |

FORMULATION 4 BLEND PROPERTIES

| | | |
|---|---|---|
| Bulk Density: | 0.52 | g/cc |
| Flow Rate Index: | 2.86 | kg/sec |
| Tap Density: | 0.66 | g/cc |

Sieve Analysis

| | |
|---|---|
| Mesh Size/ | Mesh Size/ |
| % Retained | % Retained |

| | | | |
|---|---|---|---|
| 40 | 23.6% | 80 | 33.2% |
| 100 | 6.8% | 140 | 10.5% |
| 200 | 7.6% | 325 | 8.6% |
| Pan | 9.7% | | |

FORMULATION 4 TABLET PROPERTIES

| | |
|---|---|
| Weight Variation: | 0.604 g (0.75% RSD) |
| Hardness: | 14.4 kp (5.3% RSD) |
| Thickness: | 5.42 mm (0.69% RSD) |
| Disintegration (min:sec) | 10:42, 11:00, 11:15 |
| Friability | 100 revs. 0.3509% loss |
| | 400 revs. 1.0461% loss |

FORMULATION 4 COATED TABLET DISSOLUTION
Actual Film Coat 1.6% by weight

| 15 min | 30 min | 45 min | 60 min | 90 min |
|---|---|---|---|---|
| 63.8 (2.4%) | 96.8 (2.1%) | 98.2 (1.1%) | 98.4 (1.0%) | 98.6 (0.9%) |

### Formulation 5

Formulation 5 has a high Shear Granulation with 1.5% by weight PVP binder and 0% by weight disintegrant intra-granular, and 1.5% by weight pre-gelatinized starch extra-granular. The components of Formulation 5 are as follows:

INTRA-GRANULAR

| Component | mg/tablet | W/W % |
|---|---|---|
| (R)-2-(2-fluoro-4- | 400.00 | 66.667 |
| biphenylyl)propionic acid | | |
| Drug Substance | | |
| Lactose, Anhydrous | 71.60 | 6.933 |
| Cab-O-Sil M5P | 2.70 | 0.45 |
| PVP K29/32 | 9.00 | 1.50 |
| Purified Water | — | — |
| Total of Intra-Granular | 483.30 | 80.55 |

EXTRA-GRANULAR

| Component | mg/tablet | W/W % |
|---|---|---|
| Avicel ® PH 302 | 99.00 | 16.50 |
| Cab-O-Sil M5P | 2.70 | 0.45 |
| Pregelatinized Starch | 9.00 | 1.50 |
| Magnesium Stearate NF | 6.00 | 1.00 |
| Total | 600.00 | 100.00 |

FORMULATION 5 BLEND PROPERTIES

| | | |
|---|---|---|
| Bulk Density: | 0.56 | g/cc |
| Flow Rate Index: | 0.844 | kg/sec |
| Tap Density: | 0.74 | g/cc |

Sieve Analysis

| | |
|---|---|
| Mesh Size/ | Mesh Size/ |
| % Retained | % Retained |

| | | | |
|---|---|---|---|
| 40 | 10.5% | 80 | 33.4% |
| 100 | 6.0% | 140 | 9.6% |
| 200 | 10.0% | 325 | 13.7% |
| Pan | 16.8% | | |

FORMULATION 5 TABLET PROPERTIES

| | |
|---|---|
| Weight Variation: | 0.5992 g (0.4% RSD) |
| Hardness: | 15.8 kp (5.4% RSD) |
| Thickness: | 5.31 mm (0.4% RSD) |
| Disintegration (min:sec): | 37:15, 38:22, 38:29 |
| Friability: | 100 rev. 0.33% 400 rev. 1.6% |

FORMULATION 5 COATED TABLET DISSOLUTION
Actual Film Coat 1.8%

| 15 min | 30 min | 45 min | 60 min | 90 min |
|---|---|---|---|---|
| 7.7 (27.4%) | 23.6 (19.9%) | 41.4 (24.3%) | 66.8 (28.3%) | 96.9 (2.0%) |

### Formulation 6

Formulation 6 has a high shear granulation with 6.5% by weight HPMC, 1.5% by weight disintegrant intra-granular, and 5% by weight pre-gelatinized starch extra-granular. The components of Formulation 6 are as follows:

INTRA-GRANULAR

| Component | mg/tablet | W/W % |
|---|---|---|
| (R)-2-(2-fluoro-4- | 400.00 | 66.667 |
| biphenylyl)propionic acid | | |
| drug substance | | |
| Lactose, Anhydrous | 41.60 | 6.933 |
| Cab-O-Sil M5P | 2.70 | 0.45 |
| PVP K29/32 | 9.00 | 1.50 |
| Purified Water | — | — |
| Total of Intra-Granular | 492.30 | 82.05 |

EXTRA-GRANULAR

| Component | mg/tablet | W/W % |
|---|---|---|
| Avicel ® PH 302 | 69.00 | 11.50 |
| Cab-O-Sil M5P | 2.70 | 0.45 |
| Pregelatinized Starch | 30.00 | 5.00 |
| Magnesium Stearate NF | 6.00 | 29 1.00 |
| Total | 600.00 | 100.00 |

FORMULATION 6 BLEND PROPERTIES

| | | |
|---|---|---|
| Bulk Density: | 0.47 | g/cc |
| Flow Rate Index: | 4.4 | kg/sec |
| Tap Density: | 0.59 | g/cc |

Sieve Analysis

| | |
|---|---|
| Mesh Size/ | Mesh Size/ |
| % Retained | % Retained |

| | | | |
|---|---|---|---|
| 40 | 4.1% | 80 | 27.7% |
| 100 | 10.5% | 140 | 24.5% |
| 200 | 16.6% | 325 | 11.9% |
| Pan | 4.9% | | |

FORMULATION 6 TABLET PROPERTIES

| | |
|---|---|
| Weight Variation: | 0.601 g (0.48% RSD) |
| Hardness: | 14.8 kp (5.0% RSD) |
| Thickness: | 5.35 mm (0.45% RSD) |
| Disintegration (min:sec): | 11:28, 11:40, 12:45 |
| Friability: | 100 rev. 0.33% 400 rev. 1.6% |

FORMULATION 6 COATED TABLET DISSOLUTION
Actual Film Coat 2.0%

| 15 min | 30 min | 45 min | 60 min | 90 min |
|---|---|---|---|---|
| 5.8 (22.7%) | 17.5 (21.0%) | 33.1.4 (22.4%) | 64.2 (26.7%) | 94.6 (5.6%) |

### Formulation 7

Formulation 7 has a high shear granulation with 5% by weight HPMC binder and 0% by weight disintegrant. The components of Formulation 7 are as follows:

INTRA-GRANULAR

| Component | mg/tablet | W/W % |
|---|---|---|
| (R)-2-(2-fluoro-4- | 400.00 | 66.667 |
| biphenylyl)propionic acid | | |
| drug substance | | |
| Lactose, Anhydrous | 68.60 | 11.433 |
| Cab-O-Sil M5P | 2.70 | 0.45 |
| HPMC E-5 | 30.00 | 5.00 |
| Purified Water | — | — |
| Total of Intra-Granular | 501.30 | 83.55 |

EXTRA-GRANULAR

| Component | mg/tablet | W/W % |
|---|---|---|
| Avicel ® PH 302 | 90.00 | 15.00 |
| Cab-O-Sil M5P | 2.70 | 0.45 |
| Magnesium Stearate NF | 6.00 | 1.00 |
| Total | 600.00 | 100.00 |

FORMULATION 7 BLEND PROPERTIES

| | | |
|---|---|---|
| Bulk Density: | 0.54 | g/cc |
| Flow Rate Index: | 3.14 | kg/sec |
| Tap Density: | 0.68 | g/cc |

Sieve Analysis

| | |
|---|---|
| Mesh Size/% Retained | Mesh Size/% Retained |

| | | | |
|---|---|---|---|
| 40 | 22.8% | 80 | 37.2% |
| 100 | 6.8% | 140 | 9.9% |
| 200 | 6.9% | 325 | 8.9% |
| Pan | 7.4% | | |

FORMULATION 7 TABLET PROPERTIES

| | |
|---|---|
| Weight Variation: | 0.604 g (0.793% RSD) |
| Hardness: | 14.9 kp (6.3% RSD) |
| Thickness: | 5.27 mm (0.81% RSD) |
| Disintegration (min:sec): | 46:21, 51:22, 54:20 |
| Friability: | 100 rev. 0.2988% 400 rev. 0.8675% |

FORMULATION 7 COATED TABLET DISSOLUTION
Actual Film Coat 2.2%

| 15 min | 30 min | 45 min | 60 min | 90 min |
|---|---|---|---|---|
| 3.1 (21.7%) | 9.8 (40.8%) | 32.9 (50.9%) | 82.7 (12.4%) | 96.7 (3.1%) |

### Formulation 8

Formulation 8 has a high shear granulation with 8% by weight HPMC binder and 0% by weight disintegrant. The components of Formulation 8 are as follows:

INTRA-GRANULAR

| Component | mg/tablet | W/W % |
|---|---|---|
| (R)-2-(2-fluoro-4- | 400.00 | 66.667 |
| biphenylyl)propionic acid | | |
| drug substance | | |
| Lactose, Anhydrous | 56.60 | 9.433 |
| Cab-O-Sil M5P | 2.70 | 0.45 |
| HPMC E-5 | 48.00 | 8.00 |
| Purified Water | — | — |
| Total of Intra-Granular | 507.30 | 84.55 |

EXTRA-GRANULAR

| Component | mg/tablet | W/W % |
|---|---|---|
| Avicel ® PH 302 | 84.00 | 14.00 |
| Cab-O-Sil M5P | 2.70 | 0.45 |
| Magnesium Stearate NF | 6.00 | 1.00 |
| Total | 600.00 | 100.00 |

FORMULATION 8 BLEND PROPERTIES

| | | |
|---|---|---|
| Bulk Density: | 0.56 | g/cc |
| Flow Rate Index: | 1.78 | kg/sec |
| Tap Density: | 0.71 | g/cc |

Sieve Analysis

| | |
|---|---|
| Mesh Size/% Retained | Mesh Size/% Retained |

| | | | |
|---|---|---|---|
| 40 | 30.0% | 80 | 26.8% |
| 100 | 6.6% | 140 | 9.5% |
| 200 | 7.4% | 325 | 9.2% |
| Pan | 19.4% | | |

FORMULATION 8 TABLET PROPERTIES

| | |
|---|---|
| Weight Variation: | 0.602 g (0.398% RSD) |
| Hardness: | 14.6 kp (4.1% RSD) |
| Thickness: | 5.33 mm (0.1% RSD) |
| Disintegration (min:sec): | 52:31, 54:50, 56:33 |
| Friability: | 100 rev. 0.3099% 400 rev. 0.8804% |

FORMULATION 8 COATED TABLET DISSOLUTION
Actual Film Coat 2.3%

| 15 min | 30 min | 45 min | 60 min | 90 min |
|---|---|---|---|---|
| 5.8 (22.7%) | 17.5 (21.0%) | 33.1 (22.4%) | 64.2 (26.7%) | 94.6 (5.6%) |

### Formulation 9 Reference Tablet

Core Tablet Components

| Component | mg/tablet | Percentage W/W (of core) |
|---|---|---|
| (R)-2-(2-fluoro-4- | 400 | 50% |
| biphenylyl)propionic acid | | |
| Microcrystalline Cellulose | 392 | 49% |
| (Avicel ® 102) | | |
| Colloidal Silicon Dioxide | 4 | 0.5% |
| (Cab-O-Sil M5) | | |
| Magnesium Stearate | 4 | 0.5% |
| Total | 800 | 100 |

Coated Tablet

| | | Percentage (w/w) of coated |
|---|---|---|
| Component | mg/tablet | tablet |
| Core Tablet | 800 | 93% |
| Coating (Opadry-II) | 56 | 7% |
| Total | 856 | 100% |

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

What is claimed is:

1. A unit dosage form comprising from 55-90% by weight (R)-2-(2-fluoro-4-biphenylyl)propionic acid, from 10-45% by weight excipient, wherein said unit dosage form has 200 mg or more of (R)-2-(2-fluoro-4-biphenylyl)propionic acid.

2. The unit dosage form of claim 1 wherein said (R)-2-(2-fluoro-4-biphenylyl)propionic acid is the free acid form of (R)-2-(2-fluoro-4-biphenylyl)propionic acid.

3. The unit dosage form of claim 1 wherein said dosage form is a tablet or a capsule.

4. The unit dosage form of claim 1 comprising from 380-420 mg (R)-2-(2-fluoro-4-biphenylyl)propionic acid and from 200-260 mg excipient.

5. The unit dosage form of claim 4 wherein when orally administered to a fasting subject provides a Cₘₐₓ of from about 30 to about 95 μg/ml.

6. The unit dosage form of claim 5 which is suitable for oral administration.

7. The unit dosage form of claim 1 comprising from 55-80% by weight (R)-2-(2-fluoro-4-biphenylyl)propionic acid and from about 20-45% by weight excipient that has a total weight of less than 800 mg.

8. The unit dosage form of claim 1 which is a coated tablet.

9. The unit dosage form of claim 1 having from about 380 mg to 420 mg (R)-2-(2-fluoro-4-biphenylyl)propionic acid, from about 50 mg to 70 mg lactose, from about 3 mg to 7 mg colloidal silicon dioxide, from about 30 mg to 50 mg hydroxypropyl methylcellulose, from about 70 mg to 105 mg microcrystalline cellulose, from about 1 mg to 5 mg croscarmellose sodium, from about 4 mg to 8 mg magnesium stearate, and optional ingredients as desired.

10. The unit dosage form of claim 1 having from about 385 mg to 415 mg (R)-2-(2-fluoro-4-biphenylyl)propionic acid, from about 55 mg to 65 mg lactose, from about 3.5 mg to 6.5 mg colloidal silicon dioxide, from about 32 mg to 48 mg hydroxypropyl methylcellulose, from about 75 mg to 100 mg microcrystalline cellulose, from about 1.5 mg to 4.5 mg croscarmellose sodium, from about 4.5 mg to 7.5 mg magnesium stearate, and optional ingredients as desired.

11. The unit dosage form of claim 1 having from about 390 mg to 410 mg (R)-2-(2-fluoro-4-biphenylyl)propionic acid, from about 56 mg to 64 mg lactose, from about 4.0 mg to 6.5 mg colloidal silicon dioxide, from about 34 mg to 46 mg hydroxypropyl methylcellulose, from about 80 mg to 95 mg microcrystalline cellulose, from about 2.0 mg to 4.0 mg croscarmellose sodium, from about 5.0 mg to 7.0 mg magnesium stearate, and optional ingredients as desired.

12. The unit dosage form of claim 1 having from about 395 mg to 405 mg (R)-2-(2-fluoro-4-biphenylyl)propionic acid, from about 56 mg to 64 mg lactose, from about 4.0 mg to 6.0 mg colloidal silicon dioxide, from about 34 mg to 46 mg hydroxypropyl methylcellulose, from about 82 mg to 93 mg microcrystalline cellulose, from about 2.0 mg to 4.0 mg croscarmellose sodium, from about 5.0 mg to 7.0 mg magnesium stearate, and optional ingredients as desired.

13. The unit dosage form of claim 1 having 50-95% of the total weight as (R)-2-(2-fluoro-4-biphenylyl)propionic acid; 1-30% of the total weight as one or more diluents present; and 0.01-5% of the total weight as one or more glidants.

14. The unit dosage form of claim 13 having 60-95% of the total weight as (R)-2-(2-fluoro-4-biphenylyl)propionic acid; 3-25% of the total weight as one or more diluents present; and 0.1-5% of the total weight as one or more glidants.

15. The unit dosage form of claim 13 having 70-95% of the total weight as (R)-2-(2-fluoro-4-biphenylyl)propionic acid; 5-20% of the total weight as one or more diluents present; and 0.1-3% of the total weight as one or more glidants.

16. A composition having (R)-2-(2-fluoro-4-biphenylyl)propionic acid, one or more diluents, one or more glidants, one or more binders, one or more wetting agents and optionally, one or more additional ingredients.

17. The composition of claim 16 having from 50-90% (R)-2-(2-fluoro-4-biphenylyl)propionic acid, from 5-15% of one or more diluents, from 0.1-5% of one or more glidants, from 1-15% of one or more binders, from 5-25% of one or more wetting agents and from 1-10% of one or more optional ingredients.

18. A composition having a dried granulation component and one or more disintegrants wherein the dried granulation has (R)-2-(2-fluoro-4-biphenylyl)propionic acid, one or more diluents, one or more binders, and one or more glidants.

19. The composition of claim 18 wherein the one or more disintegrants is 1-40% of the total weight of the composition.

20. The composition of claim 18 wherein the one or more disintegrants is 5-25% of the total weight of the composition.
